(19) United States
(12) Patent Application Publication
(10) Pub. No.: US 2007/0243533 A1
(43) Pub. Date: Oct. 18, 2007

(54) PRODUCTS AND METHODS OF ORGANISM IDENTIFICATION IN NUTRITIONAL SUPPLEMENTS

(76) Inventor: Matthew Thomas Cimino, Baltimore, MD (US)

Correspondence Address: KILE GOEKJIAN REED & MCMANUS, 1200 NEW HAMPSHIRE AVE, NW, SUITE 570, WASHINGTON, DC, 20036, US

(21) Appl. No.: 11/379,052

(22) Filed: Apr. 17, 2006

(51) Int. Cl. C12Q 1/68; G06F 19/00; C07H 21/04

(52) U.S. Cl. 435/6; 536/24.3; 536/23.6; 702/20

(57) ABSTRACT

Certain plant species are used as nutritional or dietary supplements. Several specific regions within these supplements have been identified as particularly useful sources of diagnostic DNA sequences. These regions are found in the nuclear genome, the chloroplast genome and the mitochondrial genome. The sequences can be used to design nucleic acid probes and various diagnostic assays. These products and methods can be used to identify specific supplement species or even sub-species and to distinguish them from their closest relatives. The sequences can also be used to identify higher order groups, including geneses, families and others. Using these data, other probes and assay methods can be designed for use in a wide variety of organisms.

### BACKGROUND OF THE INVENTION

In the nutritional supplement industry, various organisms—often exotic and/or famous for a particular nutrient or other characteristic—are sought out, harvested from their natural state and delivered to the public in a processes or partially processed form. In this form it is difficult to identify the components of these supplements with species specific precision. Existing diagnostic procedures are difficult and often unreliable. This is especially true where mixtures of two or more distinct, but closely related organisms comprise the sample. In many commercial preparations, for example botanical dietary supplements, there are no established methods for identification of botanical elements within the preparations. This is a recognized problem in the dietary supplement industry. Legitimate suppliers seek to assure the public, and various regulatory agencies, of the value of their products and to contrast their products and those from cheaper, but perhaps less carefully manufactured sources.

Identification of Hypericum Species

An illustrative example of a common dietary supplement is St. John's Wort (Hypericum). The literature contains a great deal of general knowledge about Hypericum, as well as several unrelated studies. However, none of this has produced a means of identifying species and varieties of Hypericum in the processed pills and capsules which are sold to the public.

A detailed Hypericum taxonomic study has been published. This study provides a means to differentiate between types of Hypericum using morphological characters when the entire plant is present, though virtually nothing has been done to differentiate between samples in any of the various processed states.

In addition to the taxonomic work, Hypericum samples have been subject to a significant amount of biochemical analysis. However these methods only evaluate specific compounds and do not differentiate among species. Moreover, since the active ingredients within these samples have not been established, this information is of limited value in terms of addressing the needs of the nutritional supplement industry, particularly producers and regulators.

The Hypericum literature contains several common themes: (1) a focus on identification or differentiation of whole-plant samples rather than partial or processed samples, (2) a concern for questions such as species diversity, reproduction, and horticulture, and (3) a lack of focus on the needs of the dietary supplement industry, including commercial producers, research and development concerns, regulatory agencies.

Taxonomy and Biochemistry of Hypericum

A taxonomic review of Hypericum species has been completed, but the work is cumbersome and does not address species level relationships in the group. More than 400 species of Hypericum are recognized by the botanical community. Many of these species are difficult to diagnose using traditional morphological techniques that rely on intact specimens at reproductive life stages. This problem is further compounded when one tries to identify elements at the variety level or when the plant sample is in powdered form. Additionally, more than one species of Hypericum exists in the commercial supply chain, including the known adulterant H. maculatum, creating the problem of identifying Hypericum in mixtures.

Numerous morphological and biochemical studies have been performed in an effort to recognize and classify biochemically valuable members of the genus, but these studies have not been critically examined or subject to a comprehensive literature review. Current biochemical approaches to identifying Hypericum are confounded by the absence of knowledge about which Hypericum biomolecules should be evaluated and the fact that biochemical concentrations, thought to be invariant and thus have diagnostic utility, may instead be environmentally and genetically dependent.

Hypericin, one of several biochemical compounds present in members of Hypericum, is found in varying levels among species in the genus. More specifically, multiple accessions of H. perforatum collected from a single region have been reported to contain varying levels of hypericin. One study by the USDA suggests that the level of hypericin is determined by both genetic and environmental factors. These studies indicate that taxonomic varieties of H. perforatum may contain different levels of hypericin. Furthermore, the active compounds in H. perforatum are not fully understood. A need to repeatedly identify these biotypes or varieties and track their bio-geographic origin is essential for establishing product quality and sample provenance.

Application of Nucleic Acid Assays to Hypericum

DNA-based methods have been applied to various aspects of Hypericum biology. Restriction fragment length polymorphism (RFLP) and random amplified polymorphic DNA (RAPD) analyses have been used to investigate the reproductive biology of H. perforatum. Amplified fragment length polymorphism (AFLP) analyses have also been applied to differentiate among selected species of Hypericum for the purposes of horticultural or crop improvement. These methods demonstrate the utility of DNA-based methods in the genus Hypericum, but do not address the problem of species identification and evolutionary relatedness using nucleotide sequences and phylogenetic analysis. However, since the inception of the project, DNA sequences for multiple species of Hypericum have been deposited in Genbank. None of this deposited data, however, has been associated a suggestion of using this data in identification methods.

AFLP is a useful method of DNA characterization. Nevertheless, the successful application of AFLP technology relies on consistently producing and resolving a series of fragments of amplified DNA from an organism of interest. A characteristic banding pattern inherent to each individual or species allows for reference libraries to be established, to which unknown or test AFLP patterns are compared. In theory, an unknown species in genus X can be compared to an established reference database of AFLP patterns for genus X. The AFLP approach may be spoiled by several factors, including but not limited to, the available DNA having been degraded by age or environmental factors, insufficient DNA quantity, or the available DNA occurring as mixtures from more than one organism. These conditions lead to the formation of banding patterns that will not match reference data, making the comparison inconclusive.

In the present invention, an attempt, ultimately successful, was made to use DNA sequence data for species characterization. This method is more robust than the AFLF method and not subject to the above identified shortcomings of AFLP. DNA sequence data can be determined from single fragments of degraded DNA, low concentrations of DNA, as well as from mixtures of DNA created from more than one species.

### OBJECTS OF THE INVENTION

The following objects of the invention are merely exemplary, not exhaustive, and are not meant to limit the scope of the invention in any way.

It is a general object of the invention to provide a species specific assay for identification of all photosynthetic organisms, including plants, algae and cyanobacteria.

Another general object of the invention is a species-specific assay for all members of Embryophyta.

Still another general object of the invention is a species-specific assay for members of the class Magnoliopsida.

Another general object of the invention is a species specific assay for members of the family Hypericaceae or Clusiaceae.

An additional general object of the invention is a species specific assay for members of the genus Hypericum.

Still another object of the invention is the identification of geographically distinguishable subspecies and other varieties in cases where all, or nearly all, commercially available material is derived from a single species. In these cases, sub-species specific identification is necessary in order to measure the quality of a given commercial preparation.

An additional object of the invention is a group specific assay which will determine whether a sample contains any member of a selected group such as: all photosynthetic organisms, all Embryophyta, all Magnoliopsida, all Hypericaceae, all Clusiaceae and all Hypericum.

Still another object of the invention is a method of verification of the type of Hypericum in all stages of commercial manufacturing processes.

A further object of the invention is to identify specific species within the Hypericum genus.

Another object of the invention is the identification of geographically distinguishable subspecies and other varieties within a Hypericum species.

A still further object is a method of identification which does not require unprocessed (full-plant) samples in flowering form.

Another object of the invention is to develop low cost diagnostic methods which can be practical for users with few resources, such as small commercial producers of dietary supplements. While low cost, these methods will have the same precision as more costly methods.

A final object of the invention is a rapid characterization tool for accurately diagnosing the presence of an organism to be used for product quality standards and regulation as well as for maintaining consistency in laboratory and agricultural specimens.

### BRIEF SUMMARY OF THE INVENTION

Several specific regions within the genome of plant species have been identified as particularly useful sources of diagnostic DNA sequences. These regions include the following regions in the nuclear genome: Internal Transcribed Spacer I (ITS-1), Internal Transcribed Spacer II (ITS-2), External Transcribed Spacer (ETS), the waxy locus, and the 18S rRNA locus. In the chloroplast genome, the following loci have been identified: atpB, atpB-rbcL intergenic spacer, matK, ndhF, rbcL, rpl16, rps16, rps4, trnL-trnF intergenic spacer and trnL. In the mitochondrial genome, these loci have been identified: coxII and nad.

The invention encompasses not only the specific probes derived from these regions, but also a method of isolating other probes from any of the above described organisms. Using the methods disclosed herein, probes can be readily developed that can be used to detect a specific species in a sample. Alternately, more generic probes can be developed using various methods, for example: using more conserved genetic loci, engineering specific mismatches or by using combinations of more specific probes. Alternatively, more specific probes can be developed using these methods to identify specific sub-species such as geographically isolated varieties and man-made varieties (varieties developed by the human hand, either with traditional methods such as selective breeding or more modern genetic engineering techniques).

### BRIEF DESCRIPTION OF THE DRAWINGS

FIG. 1. A partial Hypericum ITS DNA sequence alignment.

FIG. 2. A neighbor-joining analysis of Hypericum ITS sequences showing the relative differences between the species, as well as their natural relationships.

FIG. 3. Exemplar Hypericum probe regions for differentiating close relatives of Hypericum perforatum.

FIG. 4. Exemplar data that illustrate the approach for differentiating species of herbal dietary supplements.

FIGS. 5-9 illustrate DNA sequence data extracted from Genbank and used to generate a database for identification of several commercially valuable species.

### DETAILED DESCRIPTION OF THE INVENTION

Using the methods of this invention, one can create products and services to enable regulators, producers, researchers and consumers to accurately and reliably identify different selected species in a sample. For example, a St. John's Wort species can be detected in processed and partial-plant samples. These products and services will include licensed protocols that are supported by training and certification with regular updates.

The invention addresses the unmet needs of (1) a large and growing industry, (2) the regulators charged with monitoring and supporting it, (3) the millions of consumers using the product, and (4) a large body of researchers seeking to expand our understanding of the products effects and side effects.

From an industry standpoint, the invention provides a means of improved quality control allowing suppliers to verify what they are purchasing, certify what they are selling and respond to research as discoveries are made into the both the effects and side effects of different types of Hypericum.

From a regulatory standpoint it will enable FDA, FTC, and Customs to address current needs they have specifically identified as important and not addressed by current science.

From a scientific and research perspective it will enable both general and species-specific research that is currently in demand by industry and funding agencies.

Several related embodiments are also contemplated. For example the following commercial applications: Hypericum species-specific DNA probes and hybridization kits; DNA hybridization chips; supporting cross-disciplinary reference databases; methods of characterization and identification of new species, not only Hypericum but also other species-both known and newly discovered species; a service of providing updates (both updated DNA databases and updated DNA hybridization chips and probes) as new strains are discovered and/or developed; and, methods of training for Quality Assurance/Quality Control and for providing an industry recognized certification service.

Also contemplated are various methods of development of both DNA-based assays as well as improved microscopic methods for the identification of Hypericum species and the specific characterization of varieties in H. perforatum. These assays will include real-time PCR and hybridization chip technology.

DNA Component

The development of DNA probes and markers will stem from nucleotide sequence data that will also serve as the basis for a species level phylogeny. From this DNA phylogeny we can also infer the evolutionary relationships in the genus. Evolutionary trends in morphological and biochemical features can then be better recognized in Hypericum species and varieties of H. perforatum.

Key specimens were obtained for DNA analysis and microsatellite marker development. To avoid possible complications from any potential mixtures present in commercial samples, sequence data was obtained from individually identified whole plant samples, such as herbarium specimens or living tissue.

Additional Hypericum specimens were then used in the DNA phylogeny to include most if not all of the recognized species in the genus. The microsatellite markers were applied to varieties of Hypericum perforatum and specific molecular markers from this newly generated pool of data were used to identify each species of Hypericum and varieties of H. perforatum. Use the assays and DNA probes to produce specific products are described below. The complete collection of DNA markers for each species of Hypericum will provide a basis for identifying single Hypericum elements, as well as components in a mixture of two or more different Hypericum species or mixtures containing Hypericum and non-Hypericum elements.

Probe Construction

The probes of the invention are constructed using known techniques. Probes will generally be constructed having a core hybridization region. The core hybridization regions will be a contiguous region of DNA sequence complementary to the corresponding sequence selected from the sequence data disclosed in this invention. The length of the contiguous sequence can vary. More specifically, the inventor specifically contemplates every length, starting from about eight (8) nucleotides and extending, one nucleotide at a time, right up to the total length of the region from which sequence data has been obtained. The particular length will be determined by well known principles of DNA hybridization and the particular needs of the user. For example, short oligonucleotides can be useful as hybridization probes because even single base pair mismatches can be detected. These types of probes will be useful in cases where the target DNA sequence (the sequence in the organism to be detected) differs by only one base from an exogenous sequence that can potentially interfere with the target hybridization and generate unwanted background cross-hybridization. Other preferred embodiments utilize longer core hybridization regions, for example, where a lower hybridization Tₘ is desired.

It is also specifically contemplated that for certain specific applications, mismatches can be introduced to the core hybridization region, usually only a one or two base pair mismatch, addition or deletion. For example, if a background hybridization signal cannot be eliminated with a probe having a perfect match to its target sequence, a mismatch can be introduced. This mismatch can increase the difference in Tₘ between the target and the exogenous background hybridization, thus making them easier to distinguish.

Other possible embodiments of the probes will have, in addition to the core region, detectable labels and flanking regions having functions that facilitate recombinant DNA techniques. Some examples are plasmid vector sequences, replication origins, restriction endonuclease recognition sites, primer binding sites, translation and transcription origins, multiple cloning sites and the like.

Reference Database Component

A database that contains biochemical, microscopy, and phylogenetic data will allow the user of this invention to integrate a large body of information. The inventor contemplates providing this data in a form manageable for the general public and commercial users.

Using methods described herein a database can be assembled with various formats that easily combine the markedly different types of information associated with biochemical, microscopy, and DNA data.

Also using methods of this invention, a combined identification protocol can be written including a database that provides a means for cataloging, cross-referencing, and diagnosing different elements of Hypericum. Each species of Hypericum will be recognized as useful, potentially useful, or as an adulterant for purposes of herbal supplements.

### EXAMPLE 1

More than fifty (50) Hypericum species were analyzed for three purposes: (1) to verify that nucleic acid samples of a quality suitable for sequencings can be readily obtained from a wide variety of specimens, (2) to generate and analyze nucleotide data, and (3) obtain data on DNA variation. A critical component of this study relies on our ability to isolate and purify DNA from species of Hypericum. Next, these DNA preparations were used to identify Hypericum molecular markers that are useful for determining species level relationships.

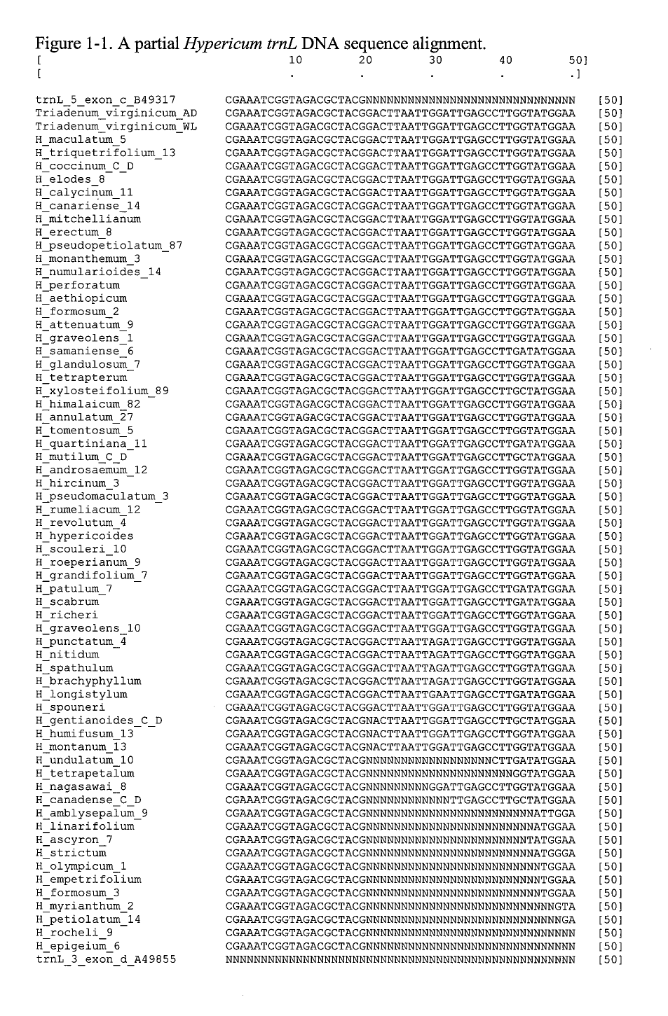

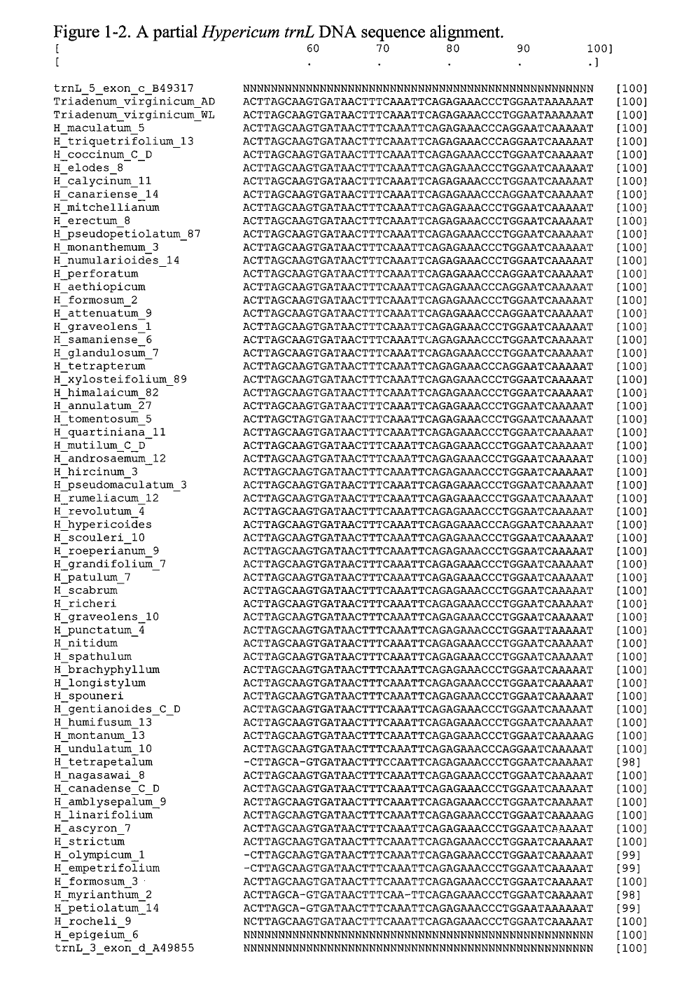

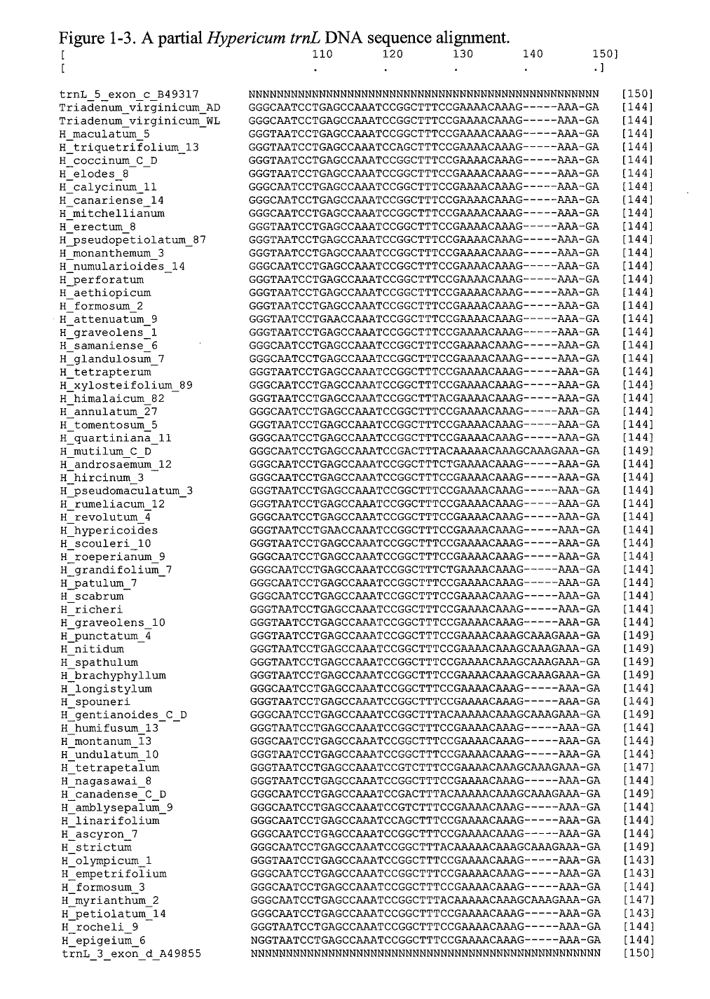

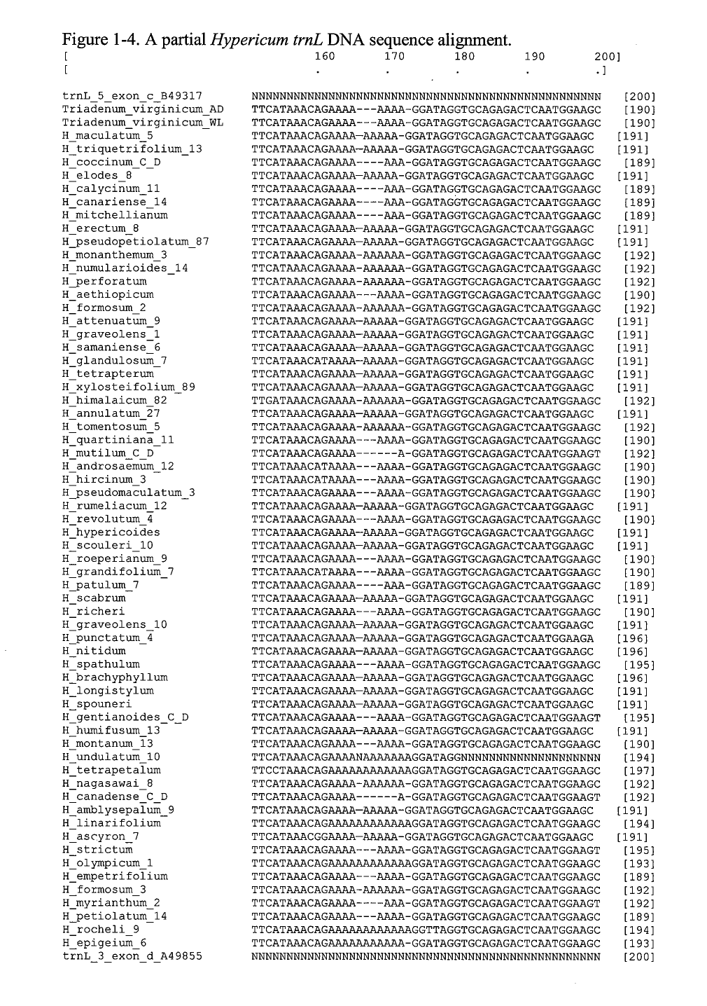

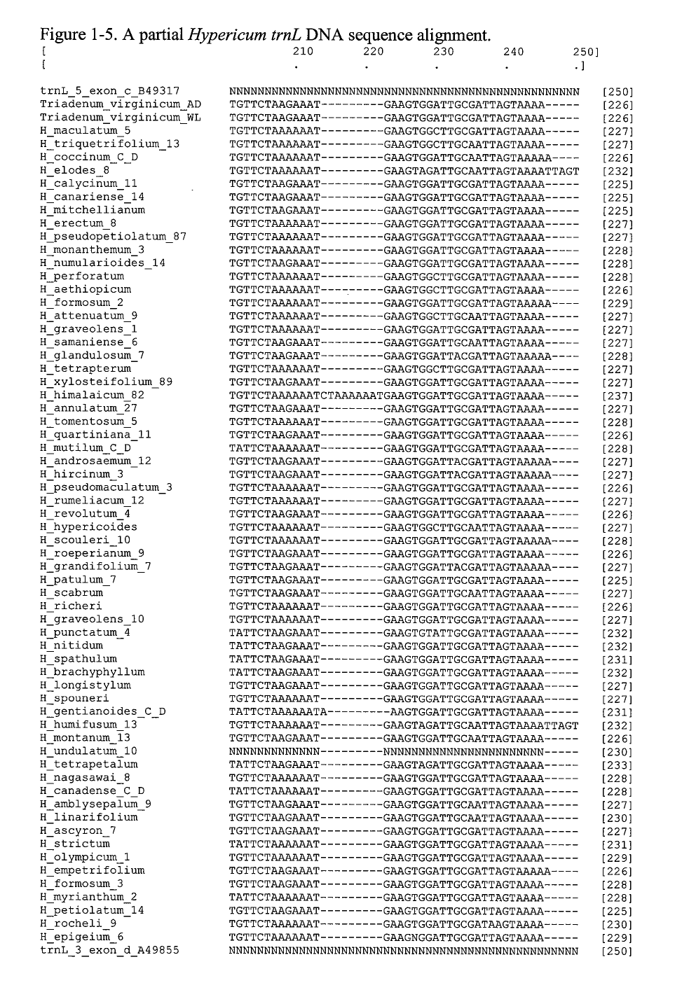

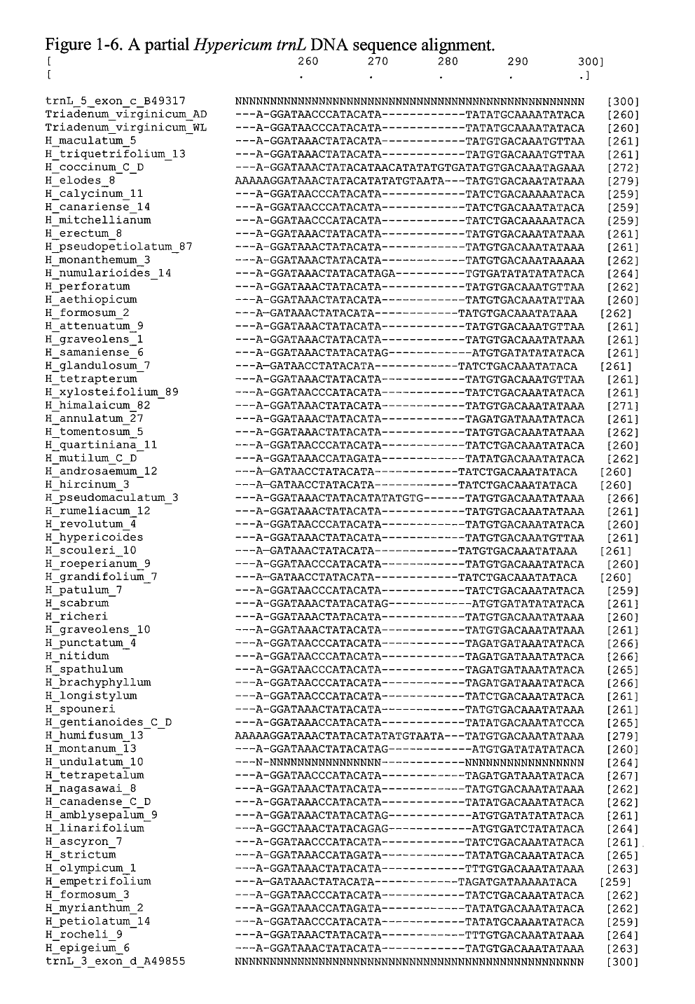

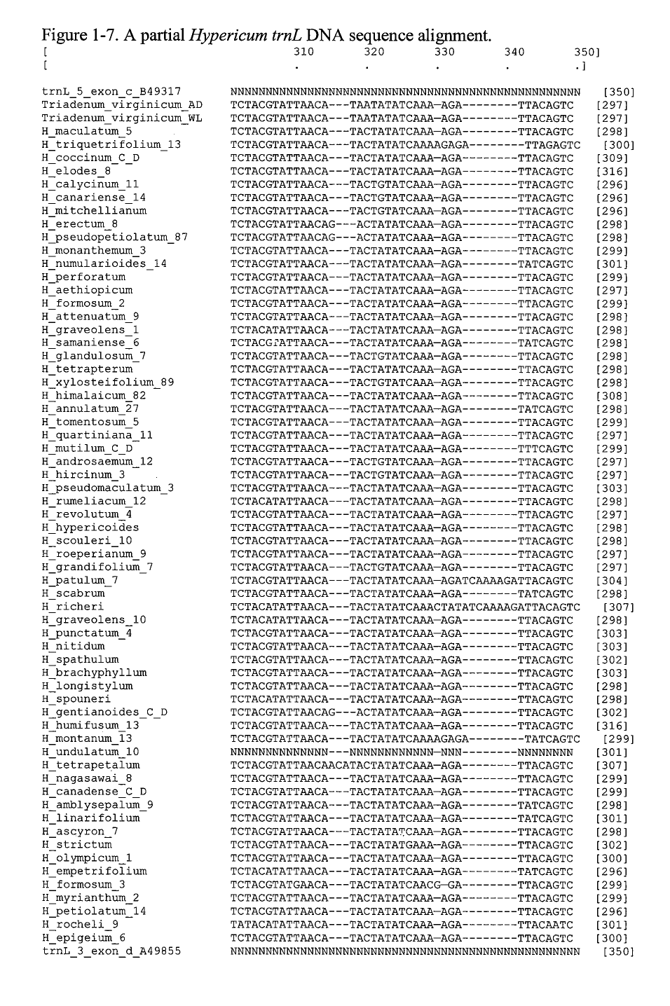

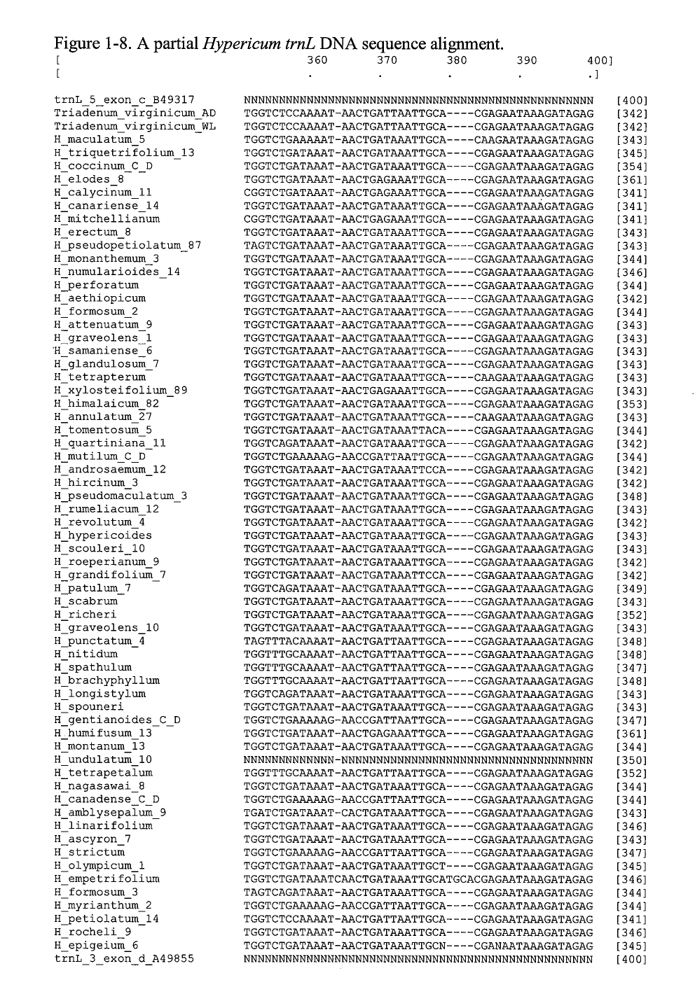

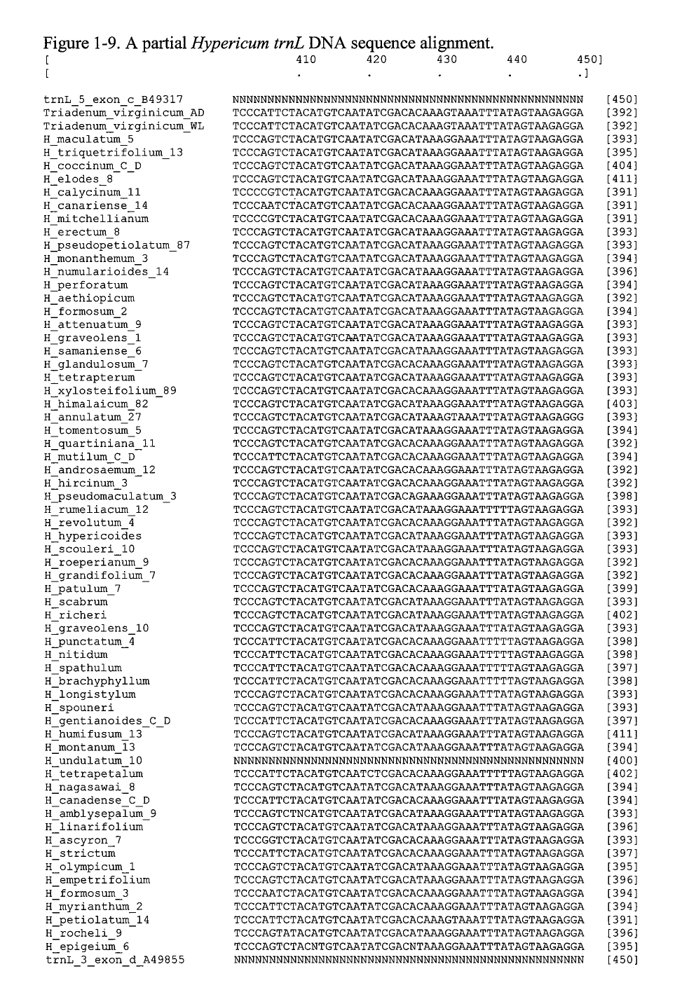

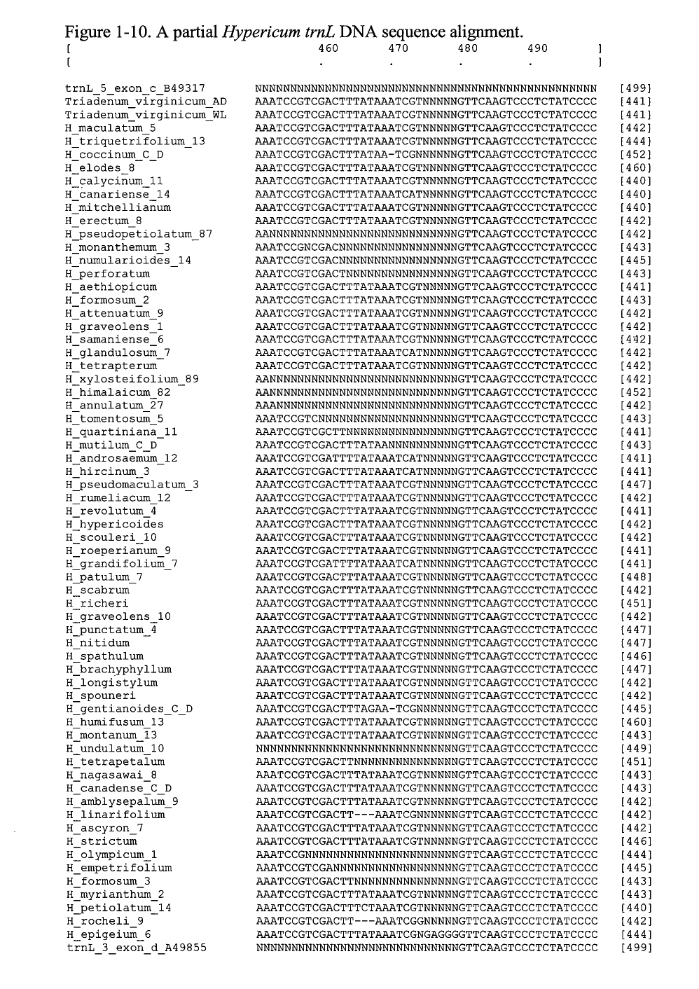

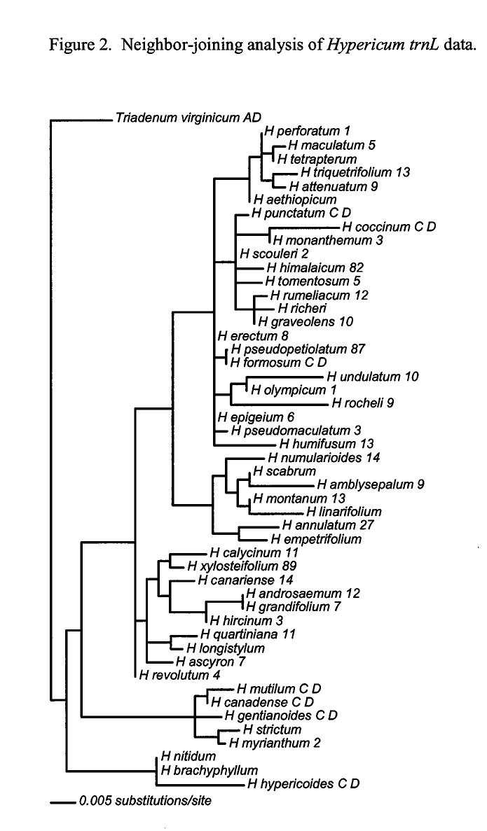

Plant tissue from more than fifty (50) species of Hypericum was obtained, DNA was isolated from these samples and specific loci were sequenced. This yielded nucleotide sequence data from the nuclear ribosomal internal transcribed spacer region (ITS) and the chloroplast trnL gene. A DNA alignment was created for the determined trnL data and the data appear to have suitable DNA variation to function in the assay methods described above. The data were determined from a region of chloroplast DNA sequence defined by the primer pairs trnL L (UAA) 5′ exon (primer C) and trnL (UAA) 3′ exon (primer D) that are described by Taberlet et al. 1991 (Universal primers for amplification of three non-coding regions of chloroplast DNA, Plant Molecular Biology, 17: 1105-1109). FIG. 1 illustrates a portion of the determined Hypericum trnL data. FIG. 2 illustrates an initial neighbor joining phylogenetic analysis performed on the trnL sequence datasets.

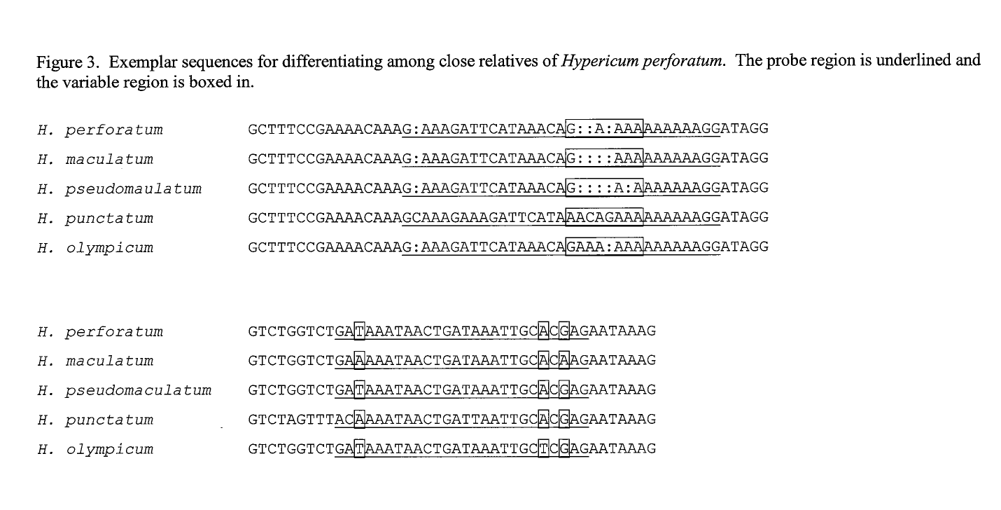

From the above data, it is a simple matter to select from the sequenced region of the various loci any of several sub-regions that have the desired property. For example, to create a species specific probe one selects a sequence found only in one of the species and not in any of the others. This example, illustrated in FIG. 3, demonstrates that the selected loci are fruitful locations within the organism's genome for finding these nucleic acid probes. This is direct evidence that the approach is enabled for the Hypericum genus. Moreover, Hypericum was an arbitrary choice, based only on the immediate commercial value of these results. Accordingly, there is every reason to believe that this method will be generally applicable. Exemplar Hypericum probe regions for differentiating close relatives of Hypericum perforatum are illustrated in FIG. 3. The probe region is underlined and the variable region is enclosed in a box.

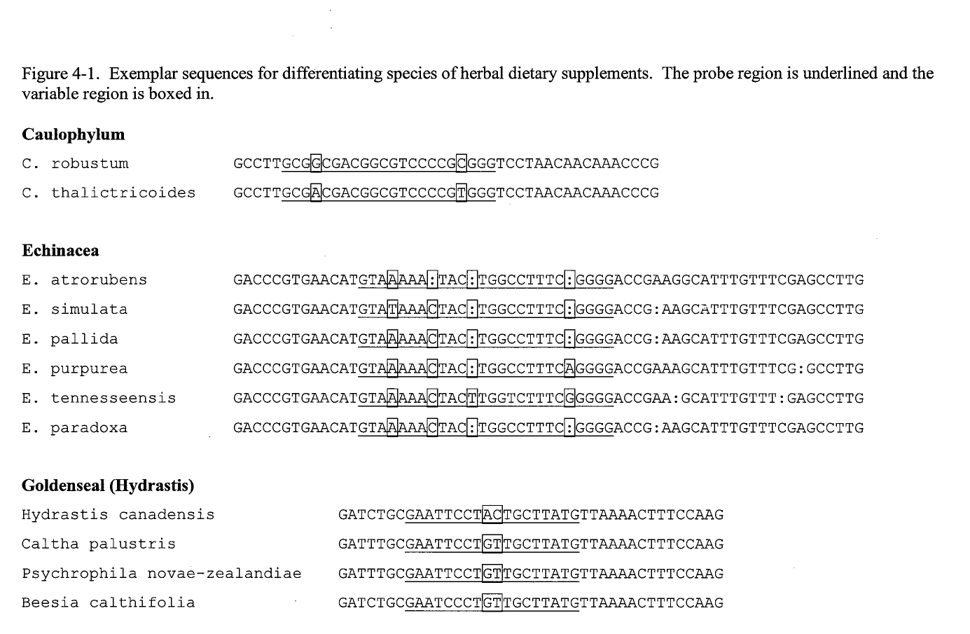

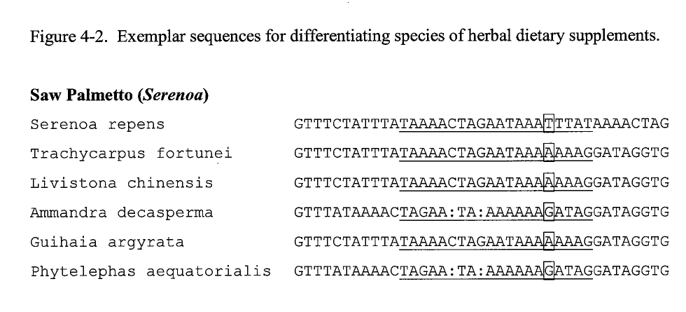

Exemplar data that illustrate the approach for differentiating species of herbal dietary supplements is illustrated in FIG. 4. The probe region is underlined and the variable region is enclosed in a box.

### EXAMPLE 2

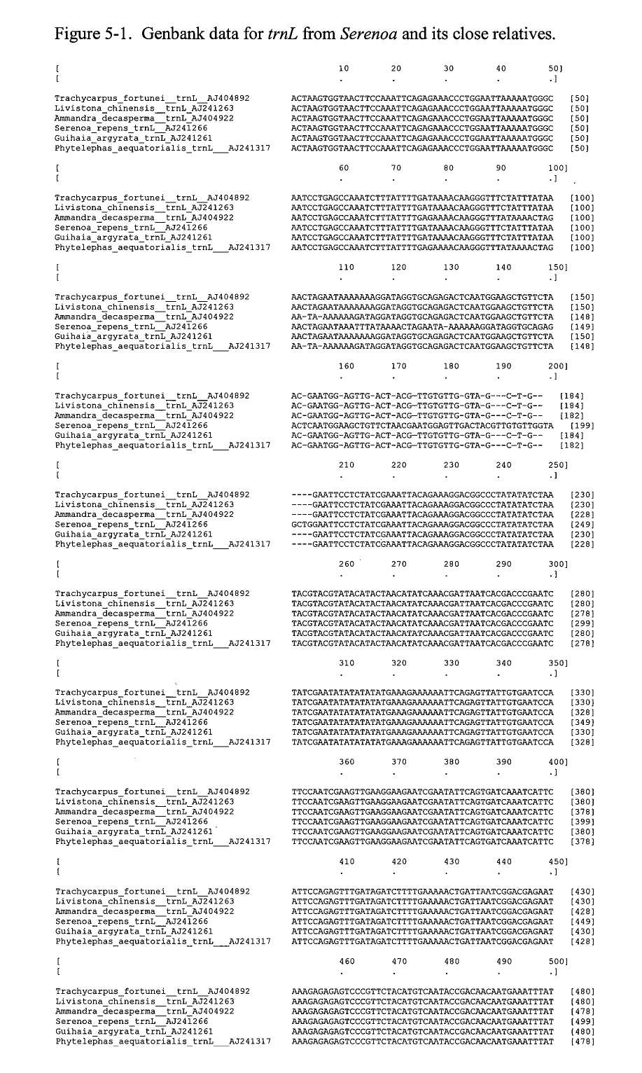

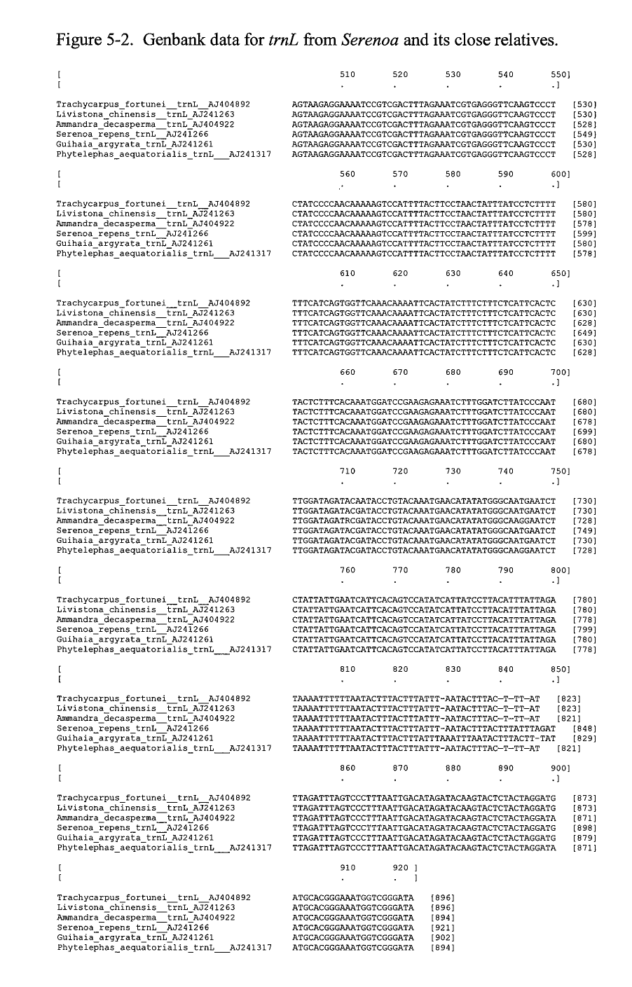

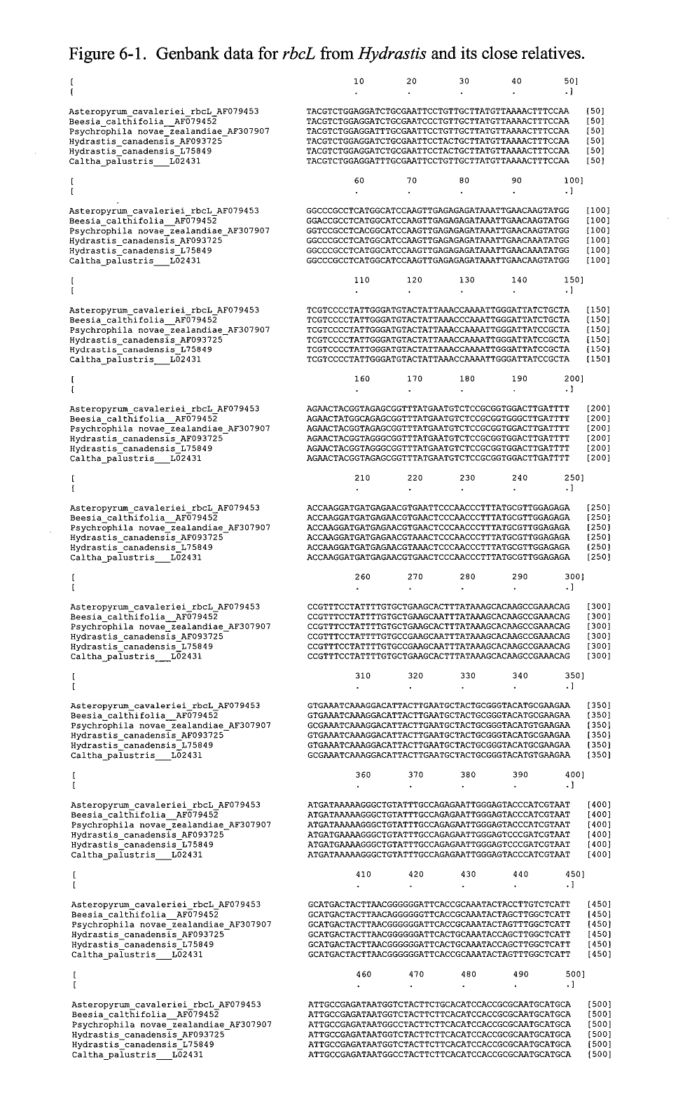

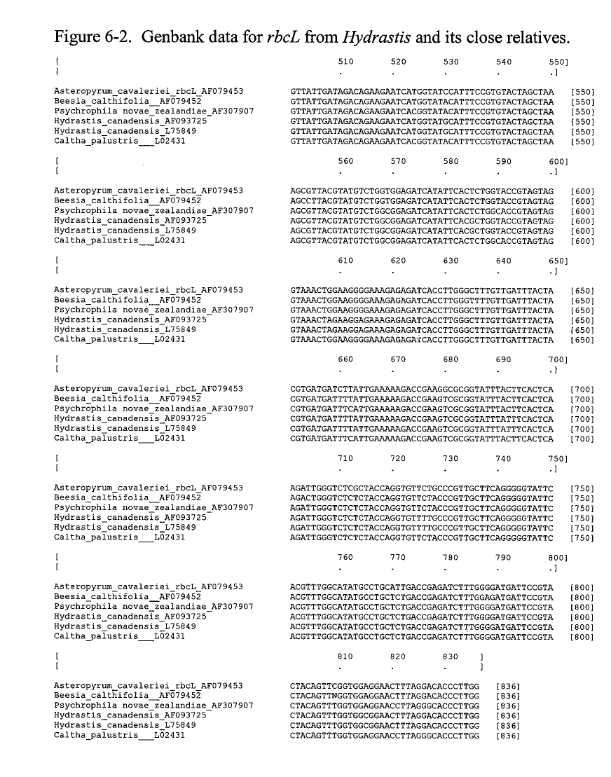

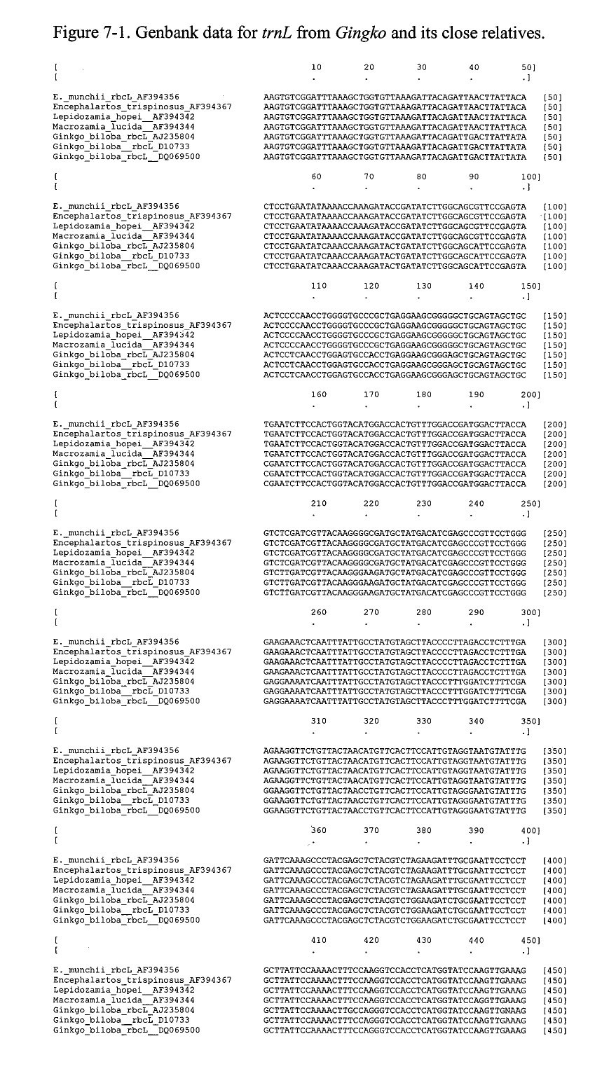

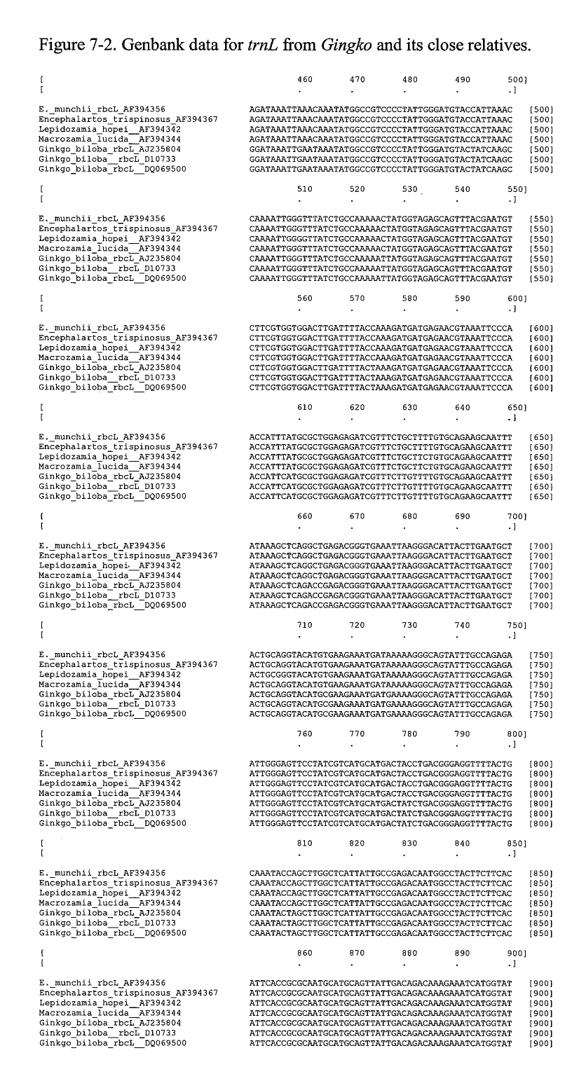

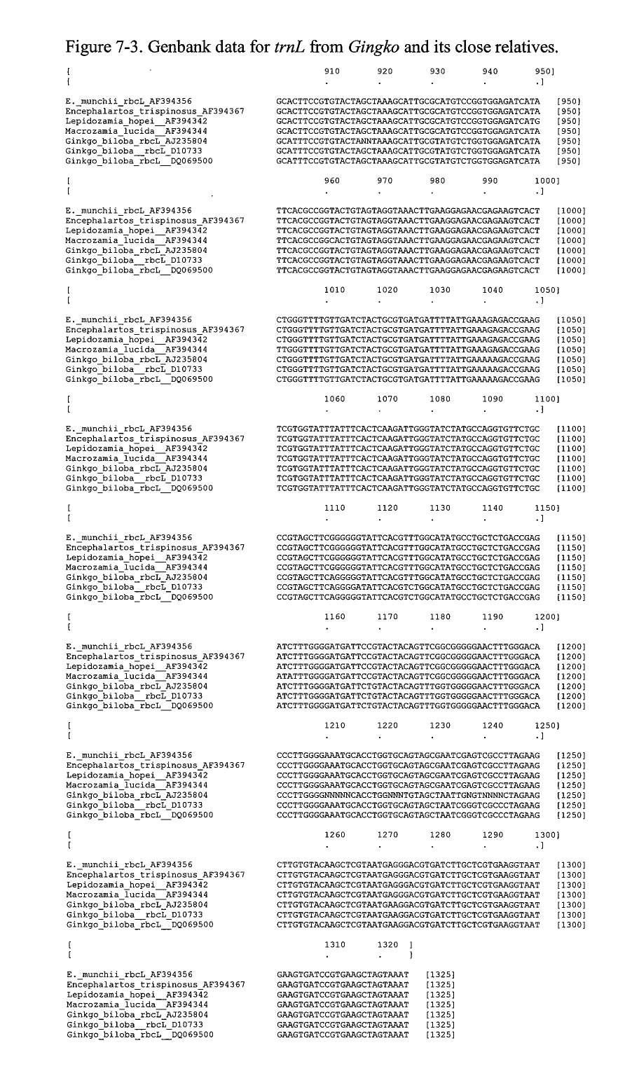

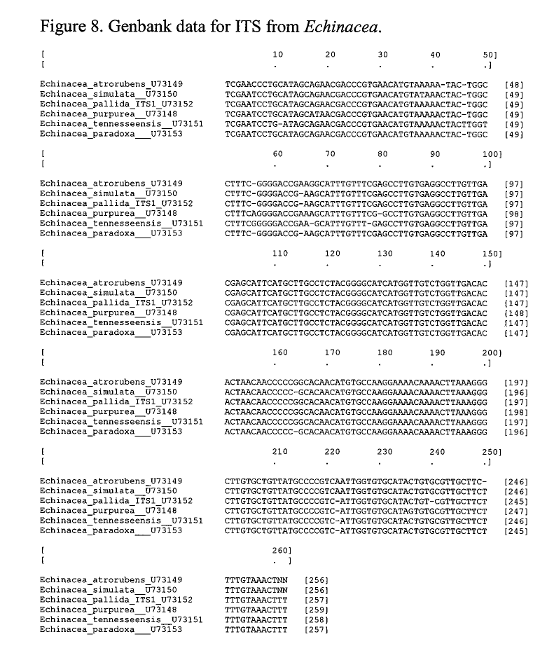

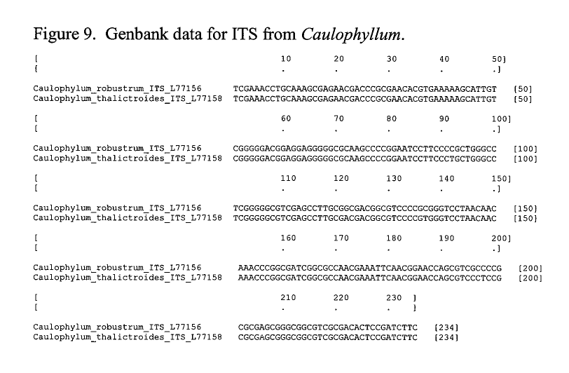

In order to further demonstrate the general usefulness of the invention, species from other genera were analyzed using a similar protocol. The following results were obtained from: Sereona (Saw Palmetto), Hydrastis (Goldenseal), Ginko, Echinacea (Coneflower), and Caulophyllum (Blue cohosh). FIG. 5 shows the data obtained from Genbank for trnL from Serenoa and its close relatives. FIG. 6 illustrates Genbank data for rbcL from Hydrastis and its close relatives. FIG. 7 shows Genbank data for rbcL from Gingko and its close relatives. FIGS. 8 and 9 illustrate Genbank data for ITS from Echinacea and Caulophyllum respectively.

Methods for the Identification of New Species

From time to time botanists discover botanical elements that are identified as being different from other botanical elements that have been previously described. Defense of these discoveries was traditionally based on distinct morphological characters that differentiate the new element from previously described botanical elements. Recently, the traditional methods have been augmented and or replaced by the use of DNA based methods that distinguish new elements from previously described elements. New species relevant to the herbal dietary supplement industry would be identified and characterized using similar morphological and DNA-based characters.

Methods of Making Species Specific Probes from Virtually any Selected Species

Variable regions of homologous DNA sequences are useful for the creation of species specific probes. Once a DNA matrix is assembled using determined DNA sequence data from the botanical elements of interest and all of their closest relatives, nucleotide character states that are specific to each botanical element can be used as hybridization sites. Complementary probes that only attached to these specific locations can be used as reporters for detecting the presence of selected botanical element. Unique nucleotide character states may consist of deletions, insertions, inversions, transitions, and transversions.

Multiple genetic loci may be screened to identify regions of DNA that are variable among the botanical elements of interest. A locus such as rbcL may be suitably variable for differentiating the monotypic genus Ginkgo from other plants, but not suitable for differentiating species in the genus Hypericum. Species specific probes can be manufactured for any photosynthetic organisms by sequencing multiple genetic loci and identifying suitably variable and uniquely variable regions in the determined data.

The comparisons made within and among species genomes also reveal certain useful properties of specific base pair positions found in these genomes. These base pair positions can all be assigned a location along a variability spectrum, from highly variable to highly conserved. This variability information is useful independent of the identity of the specific base that occupies this position. For example, this information can be used when working with newly discovered species to focus on certain loci and more quickly identify and design useful probes for the newly discovered species. Alternatively, this information can be used when studying as yet un-sequenced genomic regions of known species.

Other Products and Methods: Probe Kits, DNA Chips, DNA Databases, Etc.

A number of DNA-based products could be created from this project. These items include real-time PCR assays, nucleotide probes, microsphere assays, DNA hybridization chips, DNA databases, and any other DNA-based tool for the detection, identification, and quantification of botanical elements.

The probes can be constructed of any nucleic acid or nucleic acid analog so long as the probe specifically hybridizes to a target region. Some examples of nucleic acids and analogues are single stranded or double stranded DNA, cDNA, RNA, PNA. Also contemplated are base pairs attached to various synthetic backbones so long as the composition specifically hybridizes to a target region.

Any and all methods of detection of hybridization are contemplated, from the use of specific detectable signals associated with detectable labels attached to the probe or measuring effects associated with the hybridization itself. For example, hybridization can be detected by detecting a reaction dependent on the hybridization, for example PCR (dependent on hybridization of the primer) or a Ligase Chain Reaction (dependant on hybridization of primers to directly adjacent target regions).

### SUMMARY OF MAJOR ADVANTAGES OF THE INVENTION

The above disclosed products and methods provide a systematic protocol by which one can produce species specific nucleic acid probes. Selected regions of the target organism's genome are sequenced. These regions are located not only in the nuclear genome but also the chloroplast and mitochondrial genomes. These products and methods are applicable to all photosynthetic organisms, including plants, algae and cyanobacteria.

The resulting sequences can be analyzed and used to construct species specific probes, genus specific probes or higher taxonomic levels based on the degree of conservation of contiguous series of DNA sequences.

Alternatively, the sequences can be used to develop subspecies specific probes, or species restricted to certain geographic regions, as necessary.

At least the following specific regions, can be used to construct probes functional in the above assay; from the nuclear genome: ITS-1, ITS-2, ETS, the waxy locus, the 18S rRNA locus, the atpB; from the chloroplast genome, atpB, atpB-rbcL intergenic spacer, matK, ndhF, rbcL, rpl16, rps16, rps4, trnL-trnF intergenic spacer and trnL; and, from the mitochondrial genome: coxII and nad.

The above identified regions can be used to construct probes specific to the sub-species level, species level, members of Hypericum, Hydrastis, Serenoa and other species; specific for Hypericaceae, Clusiaceae, Magnoliopsida and Embryophyta; specific for all photosynthetic terrestrial and aquatic plants; and specific for all photosynthetic organisms including plants, algae and cyanobacteria.

In addition to the above uses, these regions can be used to monitor nutritional supplement commercial manufacturing processes and insure a high quality product.

Because of the nature of the disclosed products and assays, this invention will function with any nucleic acid containing sample. The user need not prepare and preserve unprocessed, full plant samples or flowering samples.

Also, the critical components of the disclosed assays—nucleic acid probes, kits and assays—can be produced quickly and inexpensively using the disclosed databases, yet still provide highly precise, reproducible results. Accordingly, small commercial producers can make use of the invention.

What is claimed is:

1. A Hypericum hybridization probe designed to detect a single Hypericum species in a sample comprising:
a hybridization component,
wherein said hybridization component comprises a contiguous sequence of 12 or more consecutive nucleotides selected from the group of genetic loci comprising: ITS-1, ITS-2, ETS, the waxy locus, the 18S rRNA locus, the atpB; atpB, atpB-rbcL intergenic spacer, matK, ndhF, rbcL, rpl16, rps16, rps4, tmL-trnF intergenic spacer, trnL, coxII and nad.

2. A Hypericum hybridization probe designed to detect a single Hypericum species in a sample comprising:
a hybridization component,
wherein said hybridization component comprises a contiguous sequence of 12 or more consecutive nucleotides selected from the group consisting of SEQ ID NOS 1-66.

3. A probe as defined in claim 2 wherein said hybridization component comprises a contiguous sequence of 12 or more consecutive nucleotides selected from the group of consisting of SEQ ID NOS 185-194.

4. A probe as defined in claim 1 wherein said probe is designed to differentiate close relatives of Hypericum perforatum.

5. A probe as defined in claim 4 wherein said hybridization component is selected from the group consisting of SEQ ID NOS 185-194.

6. A Caulophylum hybridization probe designed to detect a single Caulophylum species in a sample comprising:
a hybridization component,
wherein said hybridization component comprises a contiguous sequence of 12 or more consecutive nucleotides selected from the group of genetic loci comprising: ITS-1, ITS-2, ETS, the waxy locus, the 18S rRNA locus, the atpB; atpB, atpB-rbcL intergenic spacer, matK, ndhF, rbcL, rpl16, rps16, rps4, trnL-trnF intergenic spacer, trnL, coxII and nad.

7. A probe as defined in claim 6 wherein said hybridization component is selected from the group consisting of SEQ ID NOS 195-196.

8. An Echinacea hybridization probe designed to detect a single Echinacea species in a sample comprising:
a hybridization component,
wherein said hybridization component comprises a contiguous sequence of 12 or more consecutive nucleotides selected from the group of genetic loci comprising: ITS-1, ITS-2, ETS, the waxy locus, the 18S rRNA locus, the atpB; atpB, atpB-rbcL intergenic spacer, matK, ndhF, rbcL, rpl16, rps16, rps4, trnL-trnF intergenic spacer, trnL, coxII and nad.

9. A probe as defined in claim 8 wherein said hybridization component is selected from the group consisting of SEQ ID NOS 197-202.

10. A Hydrastis hybridization probe designed to detect Hydrastis from related species in a sample comprising:
a hybridization component,
wherein said hybridization component comprises a contiguous sequence of 12 or more consecutive nucleotides selected from the group of genetic loci comprising: ITS-1, ITS-2, ETS, the waxy locus, the 18S rRNA locus, the atpB; atpB, atpB-rbcL intergenic spacer, matK, ndhF, rbcL, rpl16, rps16, rps4, trnL-trnF intergenic spacer, trnL, coxII and nad.

11. A probe as defined in claim 10 wherein said hybridization component is selected from the group consisting of SEQ ID NOS 203-206.

12. A Serenoa hybridization probe designed to detect Serenoa from related species in a sample comprising:
a hybridization component,
wherein said hybridization component comprises a contiguous sequence of 12 or more consecutive nucleotides selected from the group of genetic loci comprising: ITS-1, ITS-2, ETS, the waxy locus, the 18S rRNA locus, the atpB; atpB, atpB-rbcL intergenic spacer, matK, ndhF, rbcL, rpl16, rps16, rps4, trnL-trnF intergenic spacer, trnL, coxII and nad.

13. A probe as defined in claim 12 wherein said hybridization component is selected from the group consisting of SEQ ID NOS 207-212.

14. Methods of identification of a dietary supplement comprising:
providing a sample and a nucleotide probe as defined in claims 1, 2, 3, 7, 9, 11 or 13,
mixing the sample and the probe under hybridization conditions, detecting hybridization,
wherein hybridization indicates the presence of the dietary supplement.

15. Methods of design of a hybridization probe comprising:
providing a database of nucleic acid sequences selected from one or more of the group consisting of SEQ ID NOS 1-184,
scanning the database by comparing homologous loci for mismatches,
determining whether the mismatches define a hybridization probe specific for a sub-species, species, genus, family, order, class, phylum or kingdom.


SEQUENCE LISTING (212 sequences)

SEQ ID NO: 1 - DNA, 376 bp, Hypericum perforatum_1
  repeat_region (1)..(376)
gacttaattggattgagccttggtatggaaacttagcaagtgataactttcaaattcagagaaacccaggaatcaaaaatgggtaatcctgagccaaatccggctttccgaaaacaaagaaagattcataaacagaaaaaaaaaaggataggtgcagagactcaatggaagctgttctaaaaaatgaagtggcttgcgattagtaaaaaggataaactatacatatatgtgacaaatgttaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 2 - DNA, 375 bp, Hypericum maculatum_5
  repeat_region (1)..(375)
gacttaattggattgagccttggtatggaaacttagcaagtgataactttcaaattcagagaaacccaggaatcaaaaatgggtaatcctgagccaaatccggctttccgaaaacaaagaaagattcataaacagaaaaaaaaaggataggtgcagagactcaatggaagctgttctaaaaaatgaagtggcttgcgattagtaaaaaggataaactatacatatatgtgacaaatgttaatctacgtattaacatactatatcaaaagattacagtctggtctgaaaaataactgataaattgcacaagaataaagatagagtcccagtctacatgtcaatatcgacataaaggaaatttatagtaagaggaaa

SEQ ID NO: 3 - DNA, 377 bp, Hypericum triquetrifolium_13
gacttaattggattgagccttggtatggaaacttagcaagtgataactttcaaattcagagaaacccaggaatcaaaaatgggtaatcctgagccaaatccagctttccgaaaacaaagaaagattcataaacagaaaaaaaaaggataggtgcagagactcaatggaagctgttctaaaaaatgaagtggcttgcaattagtaaaaaggataaactatacatatatgtgacaaatgttaatctacgtattaacatactatatcaaaagagattagagtctggtctgataaataactgataaattgcacgagaataaagatagagtcccagtctacatgtcaatatcgacataaaggaaatttatagtaagaggaaa

SEQ ID NO: 4 - DNA, 376 bp, Hypericum hyssopifolium_6; identical to SEQ ID NO: 1

SEQ ID NO: 5 - DNA, 374 bp, Hypericum punctatum_C_D
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaggataggtgcagagactcaatggaagctgttctaaaaaatgaagtggattgcgattagtaaaaaggagaaactatacatatatgtgacaaatataaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 6 - DNA, 386 bp, Hypericum coccinum_C_D
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaggataggtgcagagactcaatggaagctgttctaaaaaatgaagtggattgcaattagtaaaaaaggataaactatacataacatatatgtgatatgtgacaaatagaaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 7 - DNA, 393 bp, Hypericum elodes_8
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtagattgcaattagtaaaattagtaaaaaggataaactatacatatatgtaatatatgtgacaaatataaatctacgtattaacatactatatcaaaagattacagtctggtctgataaataactgagaaattgcacgagaataaagatagagtcccagtctacatgtcaatatcgacataaaggaaatttatagtaagaggaaa

SEQ ID NO: 8 - DNA, 373 bp, Hypericum calycinum_11
gacttaattggattgagccttggtatggaaacttagcaagtgataactttcaaattcagagaaaccctggaatcaaaaatgggcaatcctgagccaaatccggctttccgaaaacaaagaaagattcataaacagaaaaaaaggataggtgcagagactcaatggaagctgttctaagaaatgaagtggattgcgattagtaaaaaggataacccatacatatatctgacaaaaatacatctacgtattaacatactgtatcaaaagattacagtccggtctgataaataactgagaaattgcacgagaataaagatagagtccccgtctacatgtcaatatcgacacaaaggaaatttatagtaagaggaaa

SEQ ID NO: 9 - DNA, 373 bp, Hypericum canariense_14
gacttaattggattgagccttggtatggaaacttagcaagtgataactttcaaattcagagaaacccaggaatcaaaaatgggcaatcctgagccaaatccggctttccgaaaacaaagaaagattcataaacagaaaaaaaggataggtgcagagactcaatggaagctgttctaagaaatgaagtggattgcgattagtaaaaaggataacccatacatatatctgacaaatatacatctacgtattaacatactgtatcaaaagattacagtctggtctgataaataactgataaattgcacgagaataaagatagagtcccaatctacatgtcaatatcgacacaaaggaaatttatagtaagaggaaa

SEQ ID NO: 10 - DNA, 373 bp, Hypericum mitchellianum; identical to SEQ ID NO: 8

SEQ ID NO: 11 - DNA, 375 bp, Hypericum erectum_8
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtggattgcgattagtaaaaaggataaactatacatatatgtgacaaatataaatctacgtattaacagactatatcaaaagattacagtctggtctgataaataactgataaattgcacgagaataaagatagagtcccagtctacatgtcaatatcgacataaaggaaatttatagtaagaggaaa

SEQ ID NO: 12 - DNA, 375 bp, Hypericum pseudopetiolatum_87
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtggattgcgattagtaaaaaggataaactatacatatatgtgacaaatataaatctacgtattaacagactatatcaaaagattacagtctagtctgataaataactgataaattgcacgagaataaagatagagtcccagtctacatgtcaatatcgacataaaggaaatttatagtaagaggaaa

SEQ ID NO: 13 - DNA, 376 bp, Hypericum monanthemum_3
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaaggataggtgcagagactcaatggaagctgttctaaaaaatgaagtggattgcgattagtaaaaaggataaactatacatatatgtgacaaataaaaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 14 - DNA, 378 bp, Hypericum numularioides_14
gacttaattggattgagccttggtatggaaacttagcaagtgataactttcaaattcagagaaaccctggaatcaaaaatgggcaatcctgagccaaatccggctttccgaaaacaaagaaagattcataaacagaaaaaaaaaaggataggtgcagagactcaatggaagctgttctaagaaatgaagtggattgcgattagtaaaaaggataaactatacatagatgtgatatatatatacatctacgtattaacatactatatcaaaagatatcagtctggtctgataaataactgataaattgcacgagaataaagatagagtcccagtctacatgtcaatatcgacataaaggaaatttatagtaagaggaaa

SEQ ID NO: 15 - DNA, 376 bp, Hypericum perforatum; identical to SEQ ID NO: 1

SEQ ID NO: 16 - DNA, 375 bp, Hypericum perforatum_D
gacttaattggattgagccttggtatggaaacttagcaagtgataactttcaaattcagagaaacccaggaatcaaaaatgggtaatcctgaaccaaatccggctttccgaaaacaaagaaagattcataaacagaaaaaaaaaggataggtgcagagactcaatggaagctgttctaaaaaatgaagtggcttgcaattagtaaaaaggataaactatacatatatgtgacaaatgttaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 17 - DNA, 374 bp, Hypericum aethiopicum
gacttaattggattgagccttggtatggaaacttagcaagtgataactttcaaattcagagaaacccaggaatcaaaaatgggtaatcctgagccaaatccggctttccgaaaacaaagaaagattcataaacagaaaaaaaaggataggtgcagagactcaatggaagctgttctaaaaaatgaagtggcttgcgattagtaaaaaggataaactatacatatatgtgacaaatattaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 18 - DNA, 376 bp, Hypericum formosum_2
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaaggataggtgcagagactcaatggaagctgttctaaaaaatgaagtggattgcgattagtaaaaaagataaactatacatatatgtgacaaatataaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 19 - DNA, 375 bp, Hypericum attenuatum_9; identical to SEQ ID NO: 16

SEQ ID NO: 20 - DNA, 375 bp, Hypericum graveolens_1
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtggattgcgattagtaaaaaggataaactatacatatatgtgacaaatataaatctacatattaacatactatatcaaaagattacagtctggtctgataaataactgataaattgcacgagaataaagatagagtcccagtctacatgtcaatatcgacataaaggaaatttatagtaagaggaaa

SEQ ID NO: 21 - DNA, 375 bp, Hypericum samaniense_6
gacttaattggattgagccttgatatggaaacttagcaagtgataactttcaaattcagagaaaccctggaatcaaaaatgggcaatcctgagccaaatccggctttccgaaaacaaagaaagattcataaacagaaaaaaaaaggataggtgcagagactcaatggaagctgttctaagaaatgaagtggattgcaattagtaaaaaggataaactatacatagatgtgatatatatacatctacgtattaacatactatatcaaaagatatcagtctggtctgataaataactgataaattgcacgagaataaagatagagtcccagtctacatgtcaatatcgacataaaggaaatttatagtaagaggaaa

SEQ ID NO: 22 - DNA, 375 bp, Hypericum glandulosum_7
gacttaattggattgagccttggtatggaaacttagcaagtgataactttcaaattcagagaaaccctggaatcaaaaatgggcaatcctgagccaaatccggctttccgaaaacaaagaaagattcataaacataaaaaaaaaggataggtgcagagactcaatggaagctgttctaagaaatgaagtggattacgattagtaaaaaagataacctatacatatatctgacaaatatacatctacgtattaacatactgtatcaaaagattacagtctggtctgataaataactgataaattgcacgagaataaagatagagtcccagtctacatgtcaatatcgacacaaaggaaatttatagtaagaggaaa

SEQ ID NO: 23 - DNA, 375 bp, Hypericum tetrapterum
gacttaattggattgagccttggtatggaaacttagcaagtgataactttcaaattcagagaaacccaggaatcaaaaatgggtaatcctgagccaaatccggctttccgaaaacaaagaaagattcataaacagaaaaaaaaaggataggtgcagagactcaatggaagctgttctaaaaaatgaagtggcttgcgattagtaaaaaggataaactatacatatatgtgacaaatgttaatctacgtattaacatactatatcaaaagattacagtctggtctgataaataactgataaattgcacaagaataaagatagagtcccagtctacatgtcaatatcgacataaaggaaatttatagtaagaggaaa

SEQ ID NO: 24 - DNA, 375 bp, Hypericum xylosteifolium_89
gacttaattggattgagccttgctatggaaacttagcaagtgataactttcaaattcagagaaaccctggaatcaaaaatgggcaatcctgagccaaatccggctttccgaaaacaaagaaagattcataaacagaaaaaaaaaggataggtgcagagactcaatggaagctgttctaagaaatgaagtggattgcgattagtaaaaaggataacccatacatatatctgacaaatatacatctacgtattaacatactgtatcaaaagattacagtctggtctgataaataactgagaaattgcacgagaataaagatagagtcccagtctacatgtcaatatcgacacaaaggaaatttatagtaagaggaaa

SEQ ID NO: 25 - DNA, 385 bp, Hypericum himalaicum_82
gacttaattggattgagccttggtatggaaacttagcaagtgataactttcaaattcagagaaaccctggaatcaaaaatgggtaatcctgagccaaatccggctttacgaaaacaaagaaagattgataaacagaaaaaaaaaaggataggtgcagagactcaatggaagctgttctaaaaaatctaaaaaatgaagtggattgcgattagtaaaaaggataaactatacatatatgtgacaaatataaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 26 - DNA, 375 bp, Hypericum annulatum_27
gacttaattggattgagccttggtatggaaacttagcaagtgataactttcaaattcagagaaaccctggaatcaaaaatgggcaatcctgagccaaatccggctttccgaaaacaaagaaagattcataaacagaaaaaaaaaggataggtgcagagactcaatggaagctgttctaagaaatgaagtggattgcgattagtaaaaaggataaactatacatatagatgataaatatacatctacgtattaacatactatatcaaaagatatcagtctggtctgataaataactgataaattgcacaagaataaagatagagtcccagtctacatgtcaatatcgacataaagtaaatttatagtaagagggaa

SEQ ID NO: 27 - DNA, 376 bp, Hypericum tomentosum_5
gacttaattggattgagccttggtatggaaacttagctagtgataactttcaaattcagagaaaccctggaatcaaaaatgggtaatcctgagccaaatccggctttccgaaaacaaagaaagattcataaacagaaaaaaaaaaggataggtgcagagactcaatggaagctgttctaaaaaatgaagtggattgcgattagtaaaaaggataaactatacatatatgtgacaaatataaatctacgtattaacatactatatcaaaagattacagtctggtctgataaataactgataaattacacgagaataaagatagagtcccagtctacatgtcaatatcgacataaaggaaatttatagtaagaggaaa

SEQ ID NO: 28 - DNA, 374 bp, Hypericum quartiniana_11
gacttaattggattgagccttgatatggaaacttagcaagtgataactttcaaattcagagaaaccctggaatcaaaaatgggcaatcctgagccaaatccggctttccgaaaacaaagaaagattcataaacagaaaaaaaaggataggtgcagagactcaatggaagctgttctaagaaatgaagtggattgcgattagtaaaaaggataacccatacatatatctgacaaatatacatctacgtattaacatactatatcaaaagattacagtctggtcagataaataactgataaattgcacgagaataaagatagagtcccagtctacatgtcaatatcgacacaaaggaaatttatagtaagaggaaa

SEQ ID NO: 29 - DNA, 376 bp, Hypericum mutilum_C_D
gacttaattggattgagccttgctatggaaacttagcaagtgataactttcaaattcagagaaaccctggaatcaaaaatgggcaatcctgagccaaatccgactttacaaaaacaaagcaaagaaagattcataaacagaaaaaggataggtgcagagactcaatggaagttattctaaaaaatgaagtggattgcgattagtaaaaaggataaaccatagatatatatgacaaatatacatctacgtattaacatactatatcaaaagatttcagtctggtctgaaaaagaaccgattaattgcacgagaataaagatagagtcccattctacatgtcaatatcgacacaaaggaaatttatagtaagaggaaa

SEQ ID NO: 30 - DNA, 374 bp, Hypericum androsaemum_12
gacttaattggattgagccttggtatggaaacttagcaagtgataactttcaaattcagagaaaccctggaatcaaaaatgggcaatcctgagccaaatccggctttctgaaaacaaagaaagattcataaacataaaaaaaaggataggtgcagagactcaatggaagctgttctaagaaatgaagtggattacgattagtaaaaaagataacctatacatatatctgacaaatatacatctacgtattaacatactgtatcaaaagattacagtctggtctgataaataactgataaattccacgagaataaagatagagtcccagtctacatgtcaatatcgacacaaaggaaatttatagtaagaggaaa

SEQ ID NO: 31 - DNA, 374 bp, Hypericum hircinum_3
gacttaattggattgagccttggtatggaaacttagcaagtgataactttcaaattcagagaaaccctggaatcaaaaatgggcaatcctgagccaaatccggctttccgaaaacaaagaaagattcataaacataaaaaaaaggataggtgcagagactcaatggaagctgttctaagaaatgaagtggattacgattagtaaaaaagataacctatacatatatctgacaaatatacatctacgtattaacatactgtatcaaaagattacagtctggtctgataaataactgataaattgcacgagaataaagatagagtcccagtctacatgtcaatatcgacacaaaggaaatttatagtaagaggaaa

SEQ ID NO: 32 - DNA, 380 bp, Hypericum pseudomaculatum_3
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaggataggtgcagagactcaatggaagctgttctaaaaaatgaagtggattgcgattagtaaaaaggataaactatacatatatgtgtatgtgacaaatataaatctacgtattaacatactatatcaaaagattacagtctggtctgataaataactgataaattgcacgagaataaagatagagtcccagtctacatgtcaatatcgacagaaaggaaatttatagtaagaggaaa

SEQ ID NO: 33 - DNA, 375 bp, Hypericum rumeliacum_12
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtggattgcgattagtaaaaaggataaactatacatatatgtgacaaatataaatctacatattaacatactatatcaaaagattacagtctggtctgataaataactgataaattgcacgagaataaagatagagtcccagtctacatgtcaatatcgacataaaggaaatttttagtaagaggaaa

SEQ ID NO: 34 - DNA, 374 bp, Hypericum revolutum_4
gacttaattggattgagccttggtatggaaacttagcaagtgataactttcaaattcagagaaaccctggaatcaaaaatgggcaatcctgagccaaatccggctttccgaaaacaaagaaagattcataaacagaaaaaaaaggataggtgcagagactcaatggaagctgttctaagaaatgaagtggattgcgattagtaaaaaggataacccatacatatatgtgacaaatatacatctacgtattaacatactatatcaaaagattacagtctggtctgataaataactgataaattgcacgagaataaagatagagtcccagtctacatgtcaatatcgacacaaaggaaatttatagtaagaggaaa

SEQ ID NO: 35 - DNA, 375 bp, Hypericum hypericoides; identical to SEQ ID NO: 16

SEQ ID NO: 36 - DNA, 375 bp, Hypericum scouleri_10
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtggattgcgattagtaaaaaagataaactatacatatatgtgacaaatataaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 37 - DNA, 374 bp, Hypericum roeperianum_9; identical to SEQ ID NO: 34

SEQ ID NO: 38 - DNA, 374 bp, Hypericum grandifolium_7; identical to SEQ ID NO: 30

SEQ ID NO: 39 - DNA, 381 bp, Hypericum patulum_7
gacttaattggattgagccttgatatggaaacttagcaagtgataactttcaaattcagagaaaccctggaatcaaaaatgggcaatcctgagccaaatccggctttccgaaaacaaagaaagattcataaacagaaaaaaaggataggtgcagagactcaatggaagctgttctaagaaatgaagtggattgcgattagtaaaaaggataacccatacatatatctgacaaatatacatctacgtattaacatactatatcaaaagatcaaaagattacagtctggtcagataaataactgataaattgcacgagaataaagatagagtcccagtctacatgtcaatatcgacacaaaggaaatttatagtaagaggaaa

SEQ ID NO: 40 - DNA, 375 bp, Hypericum scabrum; identical to SEQ ID NO: 21

SEQ ID NO: 41 - DNA, 384 bp, Hypericum richeri
gacttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaggataggtgcagagactcaatggaagctgttctaaaaaatgaagtggattgcgattagtaaaaaggataaactatacatatatgtgacaaatataaatctacatattaacatactatatcaaactatatcaaaagattacagtctggtctgataaataactgataaattgcacgagaataaagatagagtcccagtctacatgtcaatatcgacataaaggaaatttatagtaagaggaaa

SEQ ID NO: 42 - DNA, 375 bp, Hypericum graveolens_10; identical to SEQ ID NO: 20

SEQ ID NO: 43 - DNA, 380 bp, Hypericum punctatum_4
gacttaattagattgagccttggtatggaaacttagcaagtgataactttcaaattcagagaaaccctggaattaaaaatgggtaatcctgagccaaatccggctttccgaaaacaaagcaaagaaagattcataaacagaaaaaaaaaggataggtgcagagactcaatggaagatattctaagaaatgaagtgtattgcgattagtaaaaaggataacccatacatatagatgataaatatacatctacgtattaacatactatatcaaaagattacagtctagtttacaaaataactgattaattgcacgagaataaagatagagtcccattctacatgtcaatatcgacacaaaggaaatttttagtaagaggaaa

SEQ ID NO: 44 - DNA, 380 bp, Hypericum nitidum
gacttaattagattgagccttggtatggaaacttagcaagtgataactttcaaattcagagaaaccctggaatcaaaaatgggtaatcctgagccaaatccggctttccgaaaacaaagcaaagaaagattcataaacagaaaaaaaaaggataggtgcagagactcaatggaagctattctaagaaatgaagtggattgcgattagtaaaaaggataacccatacatatagatgataaatatacatctacgtattaacatactatatcaaaagattacagtctggtttgcaaaataactgattaattgcacgagaataaagatagagtcccattctacatgtcaatatcgacacaaaggaaatttttagtaagaggaaa

SEQ ID NO: 45 - DNA, 379 bp, Hypericum spathulum
gacttaattagattgagccttggtatggaaacttagcaagtgataactttcaaattcagagaaaccctggaatcaaaaatgggtaatcctgagccaaatccggctttccgaaaacaaagcaaagaaagattcataaacagaaaaaaaaggataggtgcagagactcaatggaagctattctaagaaatgaagtggattgcgattagtaaaaaggataacccatacatatagatgataaatatacatctacgtattaacatactatatcaaaagattacagtctggtttgcaaaataactgattaattgcacgagaataaagatagagtcccattctacatgtcaatatcgacacaaaggaaatttttagtaagaggaaa

SEQ ID NO: 46 - DNA, 380 bp, Hypericum brachyphyllum; identical to SEQ ID NO: 44

SEQ ID NO: 47 - DNA, 375 bp, Hypericum longistylum
gacttaattgaattgagccttgatatggaaacttagcaagtgataactttcaaattcagagaaaccctggaatcaaaaatgggcaatcctgagccaaatccggctttccgaaaacaaagaaagattcataaacagaaaaaaaaaggataggtgcagagactcaatggaagctgttctaagaaatgaagtggattgcgattagtaaaaaggataacccatacatatatctgacaaatatacatctacgtattaacatactatatcaaaagattacagtctggtcagataaataactgataaattgcacgagaataaagatagagtcccagtctacatgtcaatatcgacacaaaggaaatttatagtaagaggaaa

SEQ ID NO: 48 - DNA, 375 bp, Hypericum spouneri; identical to SEQ ID NO: 20

SEQ ID NO: 49 - DNA, 378 bp, Hypericum gentianoides_C_D
acttaattggattgagccttgctatggaaacttagcaagtgataactttcaaattcagagaaaccctggaatcaaaaatgggcaatcctgagccaaatccggctttacaaaaacaaagcaaagaaagattcataaacagaaaaaaaaggataggtgcagagactcaatggaagttattctaaaaaataaagtggattgcgattagtaaaaaggataaaccatacatatatatgacaaatatccatctacgtattaacagactatatcaaaagattacagtctggtctgaaaaagaaccgattaattgcacgagaataaagatagagtcccattctacatgtcaatatcgacacaaaggaaatttatagtaagaggaaa

SEQ ID NO: 50 - DNA, 392 bp, Hypericum humifusum_13
acttaattggattgagccttggtatggaaacttagcaagtgataactttcaaattcagagaaaccctggaatcaaaaatgggtaatcctgagccaaatccggctttccgaaaacaaagaaagattcataaacagaaaaaaaaaggataggtgcagagactcaatggaagctgttctaaaaaatgaagtagattgcaattagtaaaattagtaaaaaggataaactatacatatatgtaatatatgtgacaaatataaatctacgtattaacatactatatcaaaagattacagtctggtctgataaataactgagaaattgcacgagaataaagatagagtcccagtctacatgtcaatatcgacataaaggaaatttatagtaagaggaaa

SEQ ID NO: 51 - DNA, 375 bp, Hypericum montanum_13
acttaattggattgagccttggtatggaaacttagcaagtgataactttcaaattcagagaaaccctggaatcaaaaaggggcaatcctgagccaaatccggctttccgaaaacaaagaaagattcataaacagaaaaaaaaggataggtgcagagactcaatggaagctgttctaagaaatgaagtggattgcaattagtaaaaaggataaactatacatagatgtgatatatatacatctacgtattaacatactatatcaaaagagatatcagtctggtctgataaataactgataaattgcacgagaataaagatagagtcccagtctacatgtcaatatcgacataaaggaaatttatagtaagaggaaa

SEQ ID NO: 52 - DNA, 384 bp, Hypericum hypericoides_C_D
cttaattagcttgagccttggtatggaaacttagcaggtgataactttccaattcagagaaaccctggaatcaaaaatgggtaatcctgagccaaatccggctttccgaaaacaaagcaaagaaagattcataaacagaaaacaaaaaaaggataggtgcagagactcaatggaagctattctaagaaatgaagtagattgcaattagtaaaaaggataacccatacatatagatgataaatatacatctacgtattaacaacatactatatcaaaagattacagtctggtttgcaaaataactgattaattgcacgagaataaagatagagtcccattctacatgtcaatatcgacacaaaggaaattttgagtaagaggaaa

SEQ ID NO: 53 - DNA, 363 bp, Hypericum tetrapetalum
ggtatggaacttagcagtgataactttccaattcagagaaaccctggaatcaaaaatgggtaatcctgagccaaatccgtctttccgaaaacaaagcaaagaaagattcctaaacagaaaaaaaaaaaaggataggtgcagagactcaatggaagctattctaagaaatgaagtagattgcgattagtaaaaaggataacccatacatatagatgataaatatacatctacgtattaacaacatactatatcaaaagattacagtctggtttgcaaaataactgattaattgcacgagaataaagatagagtcccattctacatgtcaatctcgacacaaaggaaatttttagtaagaggaaa

SEQ ID NO: 54 - DNA, 367 bp, Hypericum nagasawai_8
ggattgagccttggtatggaaacttagcaagtgataactttcaaattcagagaaaccctggaatcaaaaatgggtaatcctgagccaaatccggctttccgaaaacaaagaaagattcataaacagaaaaaaaaaaggataggtgcagagactcaatggaagctgttctaaaaaatgaagtggattgcgattagtaaaaaggataaactatacatatatgtgacaaatataaatctacgtattaacatactatatcaaaagattacagtctggtctgataaataactgataaattgcacgagaataaagatagagtcccagtctacatgtcaatatcgacataaaggaaatttatagtaagaggaaa

SEQ ID NO: 55 - DNA, 364 bp, Hypericum canadense_C_D
ttgagccttgctatggaaacttagcaagtgataactttcaaattcagagaaaccctggaatcaaaaatgggcaatcctgagccaaatccgactttacaaaaacaaagcaaagaaagattcataaacagaaaaaggataggtgcagagactcaatggaagttattctaaaaaatgaagtggattgcgattagtaaaaaggataaaccatacatatatatgacaaatatacatctacgtattaacatactatatcaaaagattacagtctggtctgaaaaagaaccgattaattgcacgagaataaagatagagtcccattctacatgtcaatatcgacacaaaggaaatttatagtaagaggaaa

SEQ ID NO: 56 - DNA, 351 bp, Hypericum amblysepalum_9
  misc_feature (309)..(309): n is a, c, g, or t
attggaacttagcaagtgataactttcaaattcagagaaaccctggaatcaaaaatgggcaatcctgagccaaatccgtctttccgaaaacaaagaaagattcataaacagaaaaaaaaaggataggtgcagagactcaatggaagctgttctaagaaatgaagtggattgcaattagtaaaaaggataaactatacatagatgtgatatatatacatctacgtattaacatactatatcaaaagatatcagtctgatctgataaatcactgataaattgcacgagaataaagatagagtcccagtctncatgtcaatatcgacataaaggaaatttatagtaagaggaaa

SEQ ID NO: 57 - DNA, 354 bp, Hypericum linarifolium
atggaaacttagcaagtgataactttcaaattcagagaaaccctggaatcaaaaaggggcaatcctgagccaaatccagctttccgaaaacaaagaaagattcataaacagaaaaaaaaaaaaggataggtgcagagactcaatggaagctgttctaagaaatgaagtggattgcaattagtaaaaaggctaaactatacagagatgtgatctatatacatctacgtattaacatactatatcaaaagatatcagtctggtctgataaataactgataaattgcacgagaataaagatagagtcccagtctacatgtcaatatcgacataaaggaaatttatagtaagaggaaa

SEQ ID NO: 58 - DNA, 352 bp, Hypericum ascyron_7
tatggaaacttagcaagtgataactttcaaattcagagaaaccctggaatcaaaaatgggcaatcctgagccaaatccggctttccgaaaacaaagaaagattcataaacggaaaaaaaaaggataggtgcagagactcaatggaagctgttctaagaaatgaagtggattgcgattagtaaaaaggataacccatacatatatctgacaaatatacatctacgtattaacatactatatcaaaagattacagtctggtctgataaataactgataaattgcacgagaataaagatagagtcccggtctacatgtcaatatcgacacaaaggaaatttatagtaagaggaaa

SEQ ID NO: 59 - DNA, 355 bp, Hypericum strictum
atgggaacttagcaagtgataactttcaaattcagagaaaccctggaatcaaaaatgggcaatcctgagccaaatccggctttacaaaaacaaagcaaagaaagattcataaacagaaaaaaaaggataggtgcagagactcaatggaagttattctaaaaaatgaagtggattgcgattagtaaaaaggataaaccatagatatatatgacaaatatacatctacgtattaacatactatatgaaaagattacagtctggtctgaaaaagaaccgattaattgcacgagaataaagatagagtcccattctacatgtcaatatcgacacaaaggaaatttatagtaagaggaaa

SEQ ID NO: 60 - DNA, 352 bp, Hypericum olympicum_1
tggaacttagcaagtgataactttcaaattcagagaaaccctggaatcaaaaatgggtaatcctgagccaaatccggctttccgaaaacaaagaaagattcataaacagaaaaaaaaaaaaggataggtgcagagactcaatggaagctgttctaaaaaatgaagtggattgcgattagtaaaaaggataaactatacatatttgtgacaaatataaatctacgtattaacatactatatcaaaagattacagtctggtctgataaataactgataaattgctcgagaataaagatagagtcccagtctacatgtcaatatcgacataaaggaaatttatagtaagaggaaa

SEQ ID NO: 61 - DNA, 353 bp, Hypericum empetrifolium
tggaacttagcaagtgataactttcaaattcagagaaaccctggaatcaaaaatgggcaatcctgagccaaatccggctttccgaaaacaaagaaagattcataaacagaaaaaaaaggataggtgcagagactcaatggaagctgttctaagaaatgaagtggattgcgattagtaaaaaagataaactatacatatagatgataaaaatacatctacatattaacatactatatcaaaagatatcagtctggtctgataaatcaactgataaattgcatgcacgagaataaagatagagtcccagtctacatgtcaatatcgacataaaggaaatttatagtaagaggaaa

SEQ ID NO: 62 - DNA, 351 bp, Hypericum formosum_3
tggaaacttagcaagtgataactttcaaattcagagaaaccctggaatcaaaaatgggcaatcctgagccaaatccggctttccgaaaacaaagaaagattcataaacagaaaaaaaaaaggataggtgcagagactcaatggaagctgttctaagaaatgaagtggattgcgattagtaaaaaggataacccatacatatatctgacaaatatacatctacgtatgaacatactatatcaacggattacagtctagtcagataaataactgataaattgcacgagaataaagatagagtcccaatctacatgtcaatatcgacacaaaggaaatttatagtaagaggaaa

SEQ ID NO: 63 - DNA, 349 bp, Hypericum myrianthum_2
gtaacttagcagtgataactttcaattcagagaaaccctggaatcaaaaatgggcaatcctgagccaaatccggctttacaaaaacaaagcaaagaaagattcataaacagaaaaaaaggataggtgcagagactcaatggaagttattctaaaaaatgaagtggattgcgattagtaaaaaggataaaccatagatatatatgacaaatatacatctacgtattaacatactatatcaaaagattacagtctggtctgaaaaagaaccgattaattgcacgagaataaagatagagtcccattctacatgtcaatatcgacacaaaggaaatttatagtaagaggaaa

SEQ ID NO: 64 - DNA, 345 bp, Hypericum petiolatum_14
gaacttagcagtgataactttcaaattcagagaaaccctggaataaaaaatgggcaatcctgagccaaatccggctttccgaaaacaaagaaagattcataaacagaaaaaaaaggataggtgcagagactcaatggaagctgttctaagaaatgaagtggattgcgattagtaaaaaggataacccatacatatatatgcaaaatatacatctacgtattaacatactatatcaaaagattacagtctggtctccaaaataactgattaattgcacgagaataaagatagagtcccattctacatgtcaatatcgacacaaagtaaatttatagtaagaggaaa

SEQ ID NO: 65 - DNA, 347 bp, Hypericum rocheli_9
cttagcaagtgataactttcaaattcagagaaaccctggaatcaaaaatgggtaatcctgagccaaatccggctttccgaaaacaaagaaagattcataaacagaaaaaaaaaaaaggttaggtgcagagactcaatggaagctgttctaaaaaatgaagtggattgcgataagtaaaaaggataaactatacatatttgtgacaaatataaatatacatattaacatactatatcaaaagattacaatctggtctgataaataactgataaattgcacgagaataaagatagagtcccagtatacatgtcaatatcgacataaaggaaatttatagtaagaggaaa

SEQ ID NO: 66 - DNA, 296 bp, Hypericum epigeium_6
  misc_feature (110)..(110): n is a, c, g, or t
  misc_feature (227)..(227): n is a, c, g, or t
  misc_feature (231)..(231): n is a, c, g, or t
  misc_feature (256)..(256): n is a, c, g, or t
  misc_feature (270)..(270): n is a, c, g, or t
ggtaatcctgagccaaatccggctttccgaaaacaaagaaagattcataaacagaaaaaaaaaaaggataggtgcagagactcaatggaagctgttctaaaaaatgaagnggattgcgattagtaaaaaggataaactatacatatatgtgacaaatataaatctacgtattaacatactatatcaaaagattacagtctggtctgataaataactgataaattgcncganaataaagatagagtcccagtctacntgtcaatatcgacntaaaggaaatttatagtaagaggaaa

SEQ ID NO: 67 - DNA, 234 bp, Caulophylum_robustrum_ITS1
tcgaaacctgcaaagcgagaacgacccgcgaacacgtgaaaaagcattgtcgggggacggaggagggggcgcaagccccggaatccttccccgctgggcctcgggggcgtcgagccttgcggcgacggcgtccccgcgggtcctaacaacaaacccggcgatcggcgccaacgaaattcaacggaaccagcgtcgccccgcgcgagcgggcggcgtcgcgacactccgatcttc

SEQ ID NO: 68 - DNA, 234 bp, Caulophylum_thalictroides_ITS1
tcgaaacctgcaaagcgagaacgacccgcgaacacgtgaaaaagcattgtcgggggacggaggagggggcgcaagccccggaatccttccctgctgggcctcgggggcgtcgagccttgcgacgacggcgtccccgtgggtcctaacaacaaacccggcgatcggcgccaacgaaattcaacggaaccagcgtccctccgcgcgagcgggcggcgtcgcgacactccgatcttc

SEQ ID NO: 69 - DNA, 253 bp, Chamaemelum_flahaultii_ITS1
tcgaaccctgcaaagcagaacgacccgtggacacgtaaaaacaaccgagctcgagtgggttaagcacttgtttgatcctctcgatgctttgtcgatgtgcacttactcgagtcctttggagtcttgtgagtgtgtcgttggcgcaataacaacccccggcacaatgtgtgccaaggaaaactaaacgtaagaaggcttgtttcatgtagccccgttcgcggtgtgctcatgggatgtggcttctttataatca

SEQ ID NO: 70 - DNA, 253 bp, Chamaemelum_scariosum_ITS1
tcgaaccctgcaaagcagaacgacccgtgaacacgtaaaaacaaccgagctcgagtgggttaagcacttgtttgatcctctcgatgctttgtcgatgtgcacttactcgactcctttggagtcttgtgagtgtgtcgttggcgcaacaacaacccccggcacaatgtgtgccaaggaaaactaaacgtaagaaggcttgtttcatgtagccccgttcgcggtgtgctcatgggatgtggcttctttataatca

SEQ ID NO: 71 - DNA, 252 bp, Chamaemelum_fuscatum_ITS1
tcgaaccctgcaaagcagaacgacccgtgaacacgtaaaaataacgagcgtcgagtggattaagcgcttgtttgatcctcttgatgctttgtcgatgtgcattcacttgagtcctttgggtccatgtgagtgtgcattggcgcaataacaacccccggcacaacgtgtgccaaggaatactaaacgtaagaaggcttgtttcatgtagcctcgttcgcggtgtgctcatgggatgtggcttctttataatca

SEQ ID NO: 72 - DNA, 253 bp, Chamaemelum_nobile_ITS1
tcgaaccctgcaaagcagaacgacccgtgaacacgtaaaaataacgagcgtcgagtggattaagcgcttgtttgatcctctcgatgctttgtcgatgtgcattcacttgagtcctttggggtcatgtgagtgtgtcattggcgcaataacaacccccggcacaacgtgtgccaaggaatactaaacgtaagaaggcttgtttcatgttgccccgttcgcggtgtgctcatgggatgtggcttctttataatca

SEQ ID NO: 73 - DNA, 607 bp, Cimicifuga_racemosa_ITS
gaacgaccgtgaacacgttaaaaaacattatgtggatcgatgaggagtgtgagctcttaatcatccattgtcgggtcatgggattgaccacggttgatcttatgctcttgtacaaacacaaaacccggcgcaattagcgtcaaggaaatcttaacggaaacagagtgtcacccatttatagttgggcgatgcttcgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcttcgatcccaaccaattttgttagttagggaacggaaattggccccccgagtcctcttttgggcacggttggcttaaatattggtcctcgacgacaatcgtcgcggtctgcggtggttgtaaactcatccccctaagacaaaataagacgcgtagccttgtcgtctaatagaccaacataacccttggaagccgtttaacggtgttcaccctgcgaccccaggtcaggcg

SEQ ID NO: 74 - DNA, 605 bp, Cimicifuga_americana_ITS
gaacgacccgtgaacacgttaaaaaacattatgtggatcgatgaggggcgtgagctcttaatcatccattgtcgggtcatgggatcgaccacggtggatcctatgctctcgtacaaacacaaaacccgacgcaattggcgtcaaggaaatcttagcggaaacagagtgttgcccatttatagtgggcgatgctgcgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcccaaccaattttgttagttagggaacggaaattggccctccgagtccttttgggcacggttggctcaaatattggtcctcgrcggcaagtgtcgtggtctgcggtggttgtaaactcatccccctaagacgaaataagacgcgtagccttgtcgtctaacggaccaacataccccttataatccgttcaacggtgttcaccctgcgaccccaggtcaggcg

SEQ ID NO: 75 - DNA, 605 bp, Cimicifuga_laciniata_ITS
gaacgacccgtgaacacgttaaaaaacattatgtggattgatgaggggcgcgagctcttaatcatccattgtcgggtcatgggatcaaccacggtggatcctatgctctcgtacaaacacaaaacccggcgcaattggcgtcaaggaaatcttagcggaaacagagtgttgcccatttatagtgggcaatgctgcgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcccaaccaattttgttagttagggaatggaaattggctctccgagtccttttgggcacggttggctcaaatattggtcctcgacggcaagtgtcgcggtctgcggtggttgtaaactcatccccctaagacgaaataagacgcgtaaccttgtcgtctaacagaccaacataacccttagaagccgttcaacggtgttcaccctgcgaccccaggtcaggcg

SEQ ID NO: 76 - DNA, 604 bp, Cimicifuga_europaea_ITS
caacgaccgtgaacacgttaaaaaacattatgtggattgaggaggagcatgagctcttaatcatccattgtcgagtcatgggatcgaccatggttgatcttatgctctcgtacaaacacaaaacccggcgcaattggcgtcaaggaaatcttagtggaaacaaagtgttgcccatttatagtgggcgatgttgcgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcctaaccaattttattagttggggaacggaaattggccccccgagtccttttgggcacggttggctcaaatattggtcctcggcggcaagtgtcgcggtctgcggtggttgtaaactcatccccctaagacaaaataagacgcgtagccttgtcgtctaacggaccaacataacccttggaagccgttcaacggtgttcaccctgcgaccccaggtcaggcg

SEQ ID NO: 77 - DNA, 604 bp, Cimicifuga_foetida_ITS
gaacgaccgtgaacacgttaaaaaacattatgtggattgaggaggagcatgagctcttaatcatccattgtcgagtcatgggatcgaccatggttgatcttatgctctcgtacaaacacaaaacccggcgcaattggcgtcaaggaaatcttagtggaaacaaagtgttgcccatttatagtgggcgatgttgcgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcctaaccaattttattagttggggaacggaaattggccccccgagtccttttgggcacggttggctcaaatattggtcctcggcggcaagtgttgcggtctgcggtggttgtaaactcatccccctaagacgaaataagacgcgtagccttgtcgtctaacggaccaacataacccttggaagccgttctacggtgttcaccctgcgaccccaggtcaggcg

SEQ ID NO: 78 - DNA, 605 bp, Cimicifuga_mairei_ITS
gaacgaccgtgaacacgttaaaaaacattatgtggattgaggaggagcatgagctcttaatcatccattgtcgagtcatgggatcgaccacggttgatcttatgctctcgtacaaacacaaaacccggcgcaattggcgtcaaggaaatcttagtggaaacaaagtgttgcccatttatagtgggcgatgctacgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcctaaccaattttattagttggggaacggaaattggccccccgagtccttttgggcacggttggctcaaatattggtcctcggcggcaagcgtcgcggtctgcggtggttgtaaactcatccccctaagacgaaataagacgcgtagccttgtcgtataacggaccaacataacccttggaagccgttcatacggtgttcaccatgtgaccccaggtcaggtg

SEQ ID NO: 79 - DNA, 605 bp, Cimicifuga_biternata_ITS
gaatgacccgtgaacatgttaaaaaacattatatggattgatgaggggcgtgagctcttaatcatccattgtcgggccatgggattgaccacagttgatcttatgttctcgtacaaacataaaacccgacgcaattggcgtcaaggaaatcttagcggaaacaaagtgtcgcccatttatagtgggcgatgctgcgaatctaatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcccaactaattttgttagttgaggaacggaaattggccccccgagtccttttgggcacggttggctcaaatattggtcctcggcggcaagcgtcgcggtctgcggtggttgtaaactcatccccctaagacgaaataagacgcgtagccttgtcgtctaagagaccaacataacccttggaagccgttcaacggtgttcaccctgcgaccccaggtcaggtg

SEQ ID NO: 80 - DNA, 604 bp, Cimicifuga_simplex_ITS
caacgaccgtgaacacgttaaaaaacattatgtggattgacgaggagcgtgagctcttaatcatccattgtcgggtcatgggatcgaccatggttgatcttatgctctcgtacaaacacaaaacccggcgcaattggcgtcaaggaaatcttagcggaaacagagtgttgcccatttatagtgggtgatgctacgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcccaaccaattttattagttggggaatggagattggccccccgagtccttttgggcacggttggctcaaatattcgtcctcggcggcaagtgtcgcggtctacggtggttgtaaactcatccccctaagacaaaataagacgggtagccttgtcgtctaacggactaacataacccttggaagccgttcaacggtgttcaccctgcgaccccaggtcaggcg

SEQ ID NO: 81 - DNA, 604 bp, Cimicifuga_dahurica_ITS
gaatgaccgtgaacacgttaaaaaatattatgtggattgatgaggagtgtgagctctaaatcatccattgtcggggcatgggatcgactatggttgatcttatgctctcgtacaaacacaaaacccggcgcaattggcgtcaaggaaatcttaacggaaatagagtgttgcccatttatagtgggtgatgctacgaatccgatatttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcccaaccaattttattaattggggaacggaaattggccccccgagtccttttgggcacggttggctcaaatattggtcctcgacggcaagtgtcgcggtctgtggtggttgtaaactcatccccctaagacaaaataagacgcgtagacttgtcgtctaaaggaccaacataacccttggaagccgttcaacggtgttcaccctgcgaccccaggtcaggcg

SEQ ID NO: 82 - DNA, 603 bp, Cimicifuga_heracleifolia_ITS
gaacgaccgtgaacacgttaaaaaatattatgtggattgacgaggagcgtgagctttaaatcatccattgtcggggcatgggatcgaccatggttgatcgtatgctctcgtacaaacacaaaacccggcgcaattggcgtcaaggaaatcttaacggaaacagagtgttgcccatttatagtgggtgatgctacgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacatagcgtcgttcccaaccaattttattaattggggaacggaaattggccccccgagtccttttgggcacggttggctcaaatattggtcctcgacggcaagtgtcgcggtctgcggtggttgtaaactcatcccctaagacaaaataagacgcgtagccttgtcgtctaacggaccaacataacccttggaagccgttcaacggtgttcaccctgcgaccccaggtcaggcg

SEQ ID NO: 83 - DNA, 603 bp, Cimicifuga_yunnanensis_ITS
aacgaccgtgaacacgttaaaaaacattatgtggattgaggaggagcatgagctcttaatcatccattgtcgagtcatgggatcgaccacggttgatcttatgctctcgtacaaacacaaaacccggcgcaattggcgtcaaggaaatcttagtggaaacaaagtgttgcccatttatagtgggcgatgctacgaatccgatacttaaacgactctcggcaacggatatctcggctcttgcatcgatgaagaacgtagcgaaatgcgatacttggtgtgaattgcagaatcccgtgaaccatcgagtctttgaacgcaagttgcgcccgaggccatttaggttgagggcacgtctgcctgggcgtcacacacagcgtcgttcctaaccaattttattagttggggaacggaaattggccccccgagtccttttgggcacggttggctcaaatattggtcctcggcggcaagcgttgcggtttgcggtggttgtaaactcatccccctaagacgaaataagacgcgtagccttgtcgtataacggaccaacataacccttggaagccgttcaacggttttcaccatgcgaccccaggtcaggcg

SEQ ID NO: 84 - DNA, 259 bp, Echinacea_purpurea
tcgaatcctgcatagcataacgacccgtgaacatgtaaaaactactggcctttcaggggaccgaaagcatttgtttcggccttgtgaggccttgttgacgagcattcatgcttgcctctacggggcatcatggttgtctggttgacacactaacaacccccggcacaacatgtgccaaggaaaacaaaacttaaagggcttgtgctgttatgccccgtcattggtgtgcatagtgtgcgttgcttcttttgtaaacttt

SEQ ID NO: 85 - DNA, 256 bp, Echinacea_atrorubens
tcgaaccctgcatagcagaacgacccgtgaacatgtaaaaatactggcctttcggggaccgaaggcatttgtttcgagccttgtgaggccttgttgacgagcattcatgcttgcctctacggggcatcatggttgtctggttgacacactaacaacccccggcacaacatgtgccaaggaaaacaaaacttaaagggcttgtgctgttatgccccgtcaattggtgtgcatactgtgcgttgcttctttgtaaact

SEQ ID NO: 86 - DNA, 256 bp, Echinacea_simulata
tcgaatcctgcatagcagaacgacccgtgaacatgtataaactactggcctttcggggaccgaagcatttgtttcgagccttgtgaggccttgttgacgagcattcatgcttgcctctacggggcatcatggttgtctggttgacacactaacaacccccgcacaacatgtgccaaggaaaacaaaacttaaagggcttgtgctgttatgccccgtcaattggtgtgcatactgtgcgttgcttcttttgtaaact

SEQ ID NO: 87 - DNA, 257 bp, Echinacea_pallida
tcgaatcctgcatagcagaacgacccgtgaacatgtaaaaactactggcctttcggggaccgaagcatttgtttcgagccttgtgaggccttgttgacgagcattcatgcttgcctctacggggcatcatggttgtctggttgacacactaacaacccccggcacaacatgtgccaaggaaaacaaaacttaaagggcttgtgctgttatgccccgtcattggtgtgcatactgtcgttgcttcttttgtaaacttt

SEQ ID NO: 88 - DNA, 258 bp, Echinacea_tennesseensis
tcgaatcctgatagcagaacgacccgtgaacatgtaaaaactacttggtctttcgggggaccgaagcatttgtttgagccttgtgaggccttgttgacgagcattcatgcttgcctctacggggcatcatggttgtctggttgacacactaacaacccccggcacaacatgtgccaaggaaaacaaaacttaaagggcttgtgctgttatgccccgtcattggtgtgcatactgtgcgttgcttcttttgtaaacttt

SEQ ID NO: 89 - DNA, 257 bp, Echinacea_paradoxa
tcgaatcctgcatagcagaacgacccgtgaacatgtaaaaactactggcctttcggggaccgaagcatttgtttcgagccttgtgaggccttgttgacgagcattcatgcttgcctctacggggcatcatggttgtctggttgacacactaacaacccccgcacaacatgtgccaaggaaaacaaaacttaaagggcttgtgctgttatgccccgtcattggtgtgcatactgtgcgttgcttcttttgtaaacttt

SEQ ID NO: 90 - DNA, 608 bp, Eleutherococcus_senticosus
tcgaaacctgcacagcagaacgacccgcgaacacgttaccataccgggcgagggacgtggggtgcgcaagttccccaagtcgcgaacccattgtcggggatcgccctcgggcggtcctcgactgaacaacgtcaccccggcgcggaatgcgccaaggaaatcaaactgaactgaacgcgtcccacccgttcgcgggctgtgggggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaaccctgcactccctcatgggagtcatgactgaggggcggatactggcctcccgtgtctcaccgtgcggttggcccaaatgtgagtccttggcgacggacgtcacgacaagtggtggttgtaaaaagccctcttctcctgtcgtgcggtggcccgtcgccagcaaaagctcatgtgaccctgttgtgccgtcctcgacgagcactccga

SEQ ID NO: 91 - DNA, 608 bp, Eleutherococcus_sessiliflorus
tcgaaacctgcacagcagaacgacccgcgaacacgttaccataccgggtgagggacgtggggtgcgcaagttccccaagtcgcaaacccatggtcggggatcgccctcggttggtcctcgactgaacaacgtcaccccggcgcggaatgcgccaaggaaattaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggctgagggcacgtctgcctgggcgtcacgcatcgcgtcgccccccaaccctgcactccctcatgggagtcatggctgaggggcggatactggcctcccgtgtctcaccgtgcggttggcccaaatgtgagtccttggcggcggacgtcacgacaagtggtggttgtaaaaagccctcttctcctgtcgtgctgtgacccgtcgtcagcaaaagctcatgtgaccctgttgtgccgtcctcgacgagcactccga

SEQ ID NO: 92 - DNA, 615 bp, Eleutherococcus_giraldii
tcgaaacctgcacagcagaacgacccgcgaacacgttaccagttaccgggcgagggacgtggggtgcgcaagttccccaagtcgcgaacccattgtcggggatcccgccctcgggcggtcctcgactgaacaacgtcacccaggcgcggaatgcgccaaggaaatagcaaactgaactgaacgcgtcccacccgttcgcgggctgtgggggcgtctttttaaaacacaaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaaccctgcactccctcatgggagtcatgactgaggggcggatactggcctcccgtgtctcaccgtgcggttggcccaaatgtgagtccttggcgacggacgtcacgacaagtggtggttgtaaaaagccctcttctcctgtcgtgcggtggcccgtcgccagcaaaagctcatgtgaccctgttgtgccgtcctcgacgagcactccga

SEQ ID NO: 93 - DNA, 609 bp, Eleutherococcus_senticosus_ITS1
tcgaaacctgcacagcagaacgacccgcgaacacgttaccataccgttcgagggacgtggtggtgcgcaagttccccaagtcgcgaacccattgtcggggattgccctcggtcggtcctcgactcgaacaacgtcaccccggcgcggaatgcgccaaggaaatcaaactgaactgaacgcgtcccaccgttcgcgggctgtgggggcgtctttctaaaacacaaacgactctcggcaacggatatctcggctctcgcgatcgatgaagaacgtacgaaatgcgatacttggtgtgaattgcagaatcccgtgaaccatcgagtctttgaacgcaagttgcgcccgaagccattaggccaagggcacgtctgcctgggcgtcacgcatcggccgccccccaaccctgcactccctcatgggagtcatgactgaggggcggatactggcctcccgtgtctcaccgtgcggttggcccaaatgtgagtccttggcgacggacagtcacgacaagtggtggttgtaaaaagccctcttctcctgtcgtacggtggcccgtcgccagaaaaagctcatgtgaccctgttgtgccgtccacgacgagcactccga

SEQ ID NO: 94 - DNA, 608 bp, Eleutherococcus_sieboldianus
tcgaaacctgcacagcagaacgacccgcgaacacgttaccataccgggcgagggacgtggggtgcgcaagttccccaagtctcgaacccatggtcggggatcgccctcgggtggtcctcgactgaacaacgtcaccccggcgcggaatgcgccaaggaaatcaaactgaaatgaacgcgtcccacccgtccgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccaaagccattaggccgagggcacgtctgcctgggcgtcacgcatcgcgtcgccccccaaccctgcactccctcatgggagtcatgactgaggggcggatactggcttcccgtgtctcaccgcgcggttggcccaaatgtgagtccttggcgacggacgtcacgacaagtggtggttgtaaaaagccctcttcttctgtcgtgcggtggcccgtcaccaacaaaagctcatgtgaccctgttgtgccgtcctcgacgagcactccga

SEQ ID NO: 95 - DNA, 608 bp, Eleutherococcus_divaricatus_f._sachunense
tcgaaacctgcacagcagaacgacccgcgaacacgttaccataccgggtgagggacgtggggtgcgcaagttccccaagtcgcaaacccatggtcggggattgccctcggttggtcctcgactgaacaacgtcaccccggcgcggaatgcgccaaggaaattaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggctgagggcacgtctgcctgggcgtcacgcatcgcgtcgccccccaaccctgcattccctcatgggagtcatggctgaggggcggatactggcctcccgtgtctcatcgtgcggttggcccaaatgtgagtccttggcggcggacgtcacgacaagtggtggttgtaaaaagccctcttctcctgtcgtgctgtgacccgtcgtcagcaaaagctcatgtgaccctgttgtgccgtcctcgacgagcactccga

SEQ ID NO: 96 - DNA, 608 bp, Eleutherococcus_sessiliflorus_f._chungbuensis; identical to SEQ ID NO: 91

SEQ ID NO: 97 - DNA, 608 bp, Eleutherococcus_seoulensis; identical to SEQ ID NO: 91

SEQ ID NO: 98 - DNA, 608 bp, Eleutherococcus_senticosus_f._inermis; identical to SEQ ID NO: 91

SEQ ID NO: 99 - DNA, 608 bp, Eleutherococcus_divaricatus_f._distigmatis
tcgaaacctgcacagcagaacgacccgcgaacacgttaccataccgggtgagggacgtggggtgcgcaagttccccaagtcgcaaacccatggtcggggatcgccctcggttggtcctcgactgaacaacgtcaccccggcgcggaatgcgccaaggaaattaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggctgagggcacgtctgcctgggcgtcacgcatcgcgtcgccccccaaccctgcactccctcatgggagtcatggctgaggggcggatactggcctcccgtgtctcaccgtgcggttggcccaaatgtgagtccttggcggcggacgtcacgacaagtggtggttgtaaaaagccctcttctcctgtcgtgctgtgacccgtcgtcagcaaaagctcatgtgaccctgttgtgccgtcctcgacgagcactctga

SEQ ID NO: 100 - DNA, 608 bp, Eleutherococcus_divaricatus_f._tristigmatis; identical to SEQ ID NO: 91

SEQ ID NO: 101 - DNA, 608 bp, Eleutherococcus_trifoliatus_f._spinifolia
tcgaaacctgcacagcagaacgacccgcgaacacgttaccataccgggtgagggacgtggggtgcgcaagttccccaagtcgcaaacccatggtcggggatcgccctcggttggtcctcgactgaacaacgtcaccccggcgcggaatgcgccaaggaaattaaactgaactgaacgcgtcccacccgttcgcgggctgtggaggcgtctttttaaaacacaaacgactctcggcaacggatatctcggctctcgcatcgacgaagaacgtagcgaaatgcgatacttggtgtgaattgcagaatcccgtgaaccatcgagtctttgaacgcaagttgcgcccgaagccattaggctgagggcacgtctgcctgggcgtcacgcatcgcgtcgccccccaaccctgcactccctcatgggagtcatggctgaggggcggatactggcctcccgtgtctcaccgtgcggttggcccaaatgtgagtccttggcggcggacgtcacgacaagtggtggttgtaaaaagcccccttctcctgtcgtgctgtgacccgtcgtcagcaaaagctcatgtgaccctgttgtgccgtcctcgacgagcactccga

SEQ ID NO: 102 - DNA, 609 bp, Eleutherococcus_divaricatus_var._chiisanensis
tcgaaacctgcacagcagaacgacccgcgaacacgttaccataccgggtgagggacgtggggtgcgaaagttccccaagtcgcgaacccatggtcggggatcgccctcgggtggtcctcgactgaacaacgtcaccccggcgcggaatgcgccaaggaaatcaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccctaaccctgcactcccctcgtgggagtcatgactgaggggcggatactggcctcccgtgtctcaccgcgcggttggcccaaatgtgagtccttggctacgggcgtcacgacaagtggtggttgtaaaaagccctcttctcctgtcgtgcggtggcccgtcgccagcaaaagctcatgcgaccctgttgtgccgtcctcgacgagcactccga

SEQ ID NO: 103 - DNA, 609 bp, Eleutherococcus_trifoliatus; identical to SEQ ID NO: 102

SEQ ID NO: 104 - DNA, 608 bp, Eleutherococcus_koreanus
tcgaaacctgcacagcagaacgacccgcgaacacgttaccataccgggtgagggacgtggggtgcaaaagttccccaagtcgcgaacccatggtcggggatcgccctcgggtggtcctcgactgaacaacgtcaccccggcgcggaatgcgccaaggaaatcaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaaccatgcactccctcacgggagtcatgactgaggggcggatactggcctcccgtgtctcaccgcgcggttggcccaaatgtgagtccttggcgacgggcgtcacgacaagtggtggttgtaaaaagccctcttctcctgtcgtgcggtggaccgtcgccatcaaaagctcatgtgaccctgttgtgccgtcctcgacgagcactccga

SEQ ID NO: 105 - DNA, 608 bp, Eleutherococcus_gracilistylus
tcgaaacctgcacagcagaacgacccgcgaacacgttaccataccgggtgagggacgtggggtgcacaagttccccaagtcgcgaacccatggtcggggatcgccctcgggtggtcctcgactgaacaacgtcaccccggcgcggaatgcgccaaggaaatcaaactgaactgaacgcgtcccacccgttcgcgggctgtggaggcgtctttttaaaacacaaacgactctcggcaacggatatctcggctctcgcatcgatgaagaacgtagcgaaatgcgatccttggtgtgaattgcagaaccccgtgaaccatcgagtctttgaacgcaagttgcgcccgaagccattaggccgagggcacgtctgcctgggcgtcacgcatcgcgtcgccccccaaccatgcactccctcacgggagtcatgactgaggggcggatactggcctcccgtgtctcaccgcgcggttggcccaaatgtgagtccttggcgacgggcgtcacgacaagtggtggttgtaaaaagccctcttctcctgtcgtgcggtggaccgtcgccatcaaaagctcatgtgaccctgttgtgccgtcctcgacgagcactccga

SEQ ID NO: 106 - DNA, 608 bp, Eleutherococcus_simonii
tcgaaacctgcacagcagaacgacccgcgaacacgttactataccgggcgagggacgcggggtgcgcaagttccccaagtcgcgaacccatggtcggggatcgccctcgggtggtcctcgactgaacaacgtcaccccggcgcggaatgcgccaaggaaataaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgvagaatcccgtgaaccatcgagtcttttgaacgcaagttgcgcccgaagccattaggccgagggcacgtctgcctgggcgtcacgcatcgcgtcgccccccaaccctgcactccctcatgggagtcatgactgaggggcggatactggcctcccgtgtctcaccgcgcggttggcccaaatgtgagtccttggcgacggacgtcacgacaagtggtggttgtaaaaagccctcttctcctgtcgtgcggtggcccgtcgccagcaaaagctcatgtgaccctgttgtgcgtcctcgacgagcactccga

SEQ ID NO: 107 - DNA, 609 bp, Eleutherococcus_trifoliatus
tcgaaacctgcacagcagaacgacccgcgaacacgttaccataccgggtgagggacgtggggtgcgaaagttccccaagtcgcgaacccatggtcggggatcgccctcgggtggtcctcgactgaacaacgtcaccccggcgcggaatgcgccaaggaaatcaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaaccctgcactcccctcgtgggagtcatgactgaggggcggatactggcctcccgtgtctcaccgcgcggttggcccaaatgtgagtccttggctacgggcgtcacgacaagtggtggttgtaaaaagccctcttctcctgtcgtgcggtggcccgtcgccagcaaaagctcatgcgaccctgttgtgccgtcctcgacgagcactccga

SEQ ID NO: 108 - DNA, 610 bp, Eleutherococcus_trifoliatus
tcgaaacctgcacagcagaacgacccgcgaacacgttaccatacccgggtgagggacgtggggtgcgaaagttccccaagtcgcgaacccatggtcggggatcgccctcgggtggtcctcgactgaacaacgtcaccccggcgcggaatgcgccaaggaaatcaaactgaactgaacgcgtcccacccgttcgcgggctgtggaggcgtctttttaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaaccctgcactcccctcgtgggagtcatgactgaggggcggatactggcctcccgtgtctcaccgcgcggttggcccaaatgtgagtccttggctacgggcgtcacgacaagtggtggttgtaaaaagccctcttctcctgtcgtgcggtggcccgtcgccagcaaaagctcatgcgaccctgttgtgccgtcctcgacgagcactccga

SEQ ID NO: 109 - DNA, 683 bp, Epimedium_sagittatum
gaacctgcggaaggatcattgtcaaaacctgcaaagcagaacgaccagcgaacttgtgaaaaacacttatgggagggacgaagggtgttaaccttgaatccttcctattgggtcacttgggacgattgtgtcgtgaaagcgacaacgacgtccctcgttgattcaaataacaactcggcgcggtctgcgccaaggaaaatcttaacggatagagcacgtctttatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttttgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtgtaaacgacattcactttgcgaccccaggtcaggcgggactacccg

SEQ ID NO: 110 - DNA, 683 bp, Epimedium_dolichostemon
gaacctgcggaaggatcattgtcaaaacctgcaaagcagaacgaccagcgaacttgtgaaaaacacttatgggagggacgaagggtgttaaccttgaatccttcctattgggtcacttgggacgattgtgtcgtgaaagcgacaacgacgtccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtgtaaacgacattcactctgcgaccccaggtcaggcgggactacccg

SEQ ID NO: 111 - DNA, 683 bp, Epimedium_franchetii
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtgtaaacgacattcactctgcgaccccaggtcaggcgggactacccg

SEQ ID NO: 112 - DNA, 683 bp, Epimedium_grandiflorum_subsp._koreanum
gaacctgcggaaggatcattgtcaaaacctgcaaagcagaacgaccagcgaacttgtgaaaaacacttatgggagggacgaagggtgctaaccctgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccattatgcctttgttctcttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccacatggacccttgcgtgtcgtgtaaacgacattcactctgcgaccccaggtcaggcgggactacccg

SEQ ID NO: 113 - DNA, 683 bp, Epimedium_grandiflorum
gaacctgcggaaggatcattgtcaaaacctgcaaagcagaacgaccagcgaacttgtgaaaaacacttatgggagggacgaagggtgctaaccctgaatccttcctactgggtcacttgggacgattgtgtcgtgaaagcgacaacgacggccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccattatgcctttgttctcttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccacatggacccttgcgtgtcgtataaacgacattcactctgcgaccccaggtcaggcgggactacccg

SEQ ID NO: 114 - DNA, 683 bp, Epimedium_sempervirens
gaacctgcggaaggatcattgtcaaaacctgcaaagcagaacgaccagcgaacttgtgaaaaacacttatgggagggacgaagggtgctaaccctgaatccttcctactgggtcacttgggacgattgtgtcgtgaaagcgacaacgacggccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccattatgcctttgttctcttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccacatggacccttgcgtgtcgtgtaaacgacattcactctgcgaccccaggtcaggcgggactacccg

SEQ ID NO: 115 - DNA, 683 bp, Epimedium_diphyllum
gaacctgcggaaggatcattgtcaaaacctgcaaagcagaacgaccagcgaacttgtgaaaaacacttatgggagggacgaagggtgctaaccctgaatccttcctactgggtcacttgggacgattgtgtcgtgaaagcgacaacgacggccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccattatgcctttgttctcttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccacatggacccttgcgtgtcgtgtaaacgacattcactctgcaaccccaggtcaggcgggactacccg

SEQ ID NO: 116 - DNA, 683 bp, Epimedium_davidii
gaacctgcggaaggatcattgtcaaaacctgcaaagcagaacgaccagagaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgtaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 117 - DNA, 683 bp, Epimedium_ogisui
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgtaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 118 - DNA, 683 bp, Epimedium_chlorandrum
gaacctgcggaaggatcattgtcaaaacctgcacagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 119 - DNA, 683 bp, Epimedium_wushanense
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 120 - DNA, 683 bp, Epimedium_brachyrrhizum
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacggcgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 121 - DNA, 683 bp, Epimedium_epsteinii; identical to SEQ ID NO: 119

SEQ ID NO: 122 - DNA, 683 bp, Epimedium_acuminatum; identical to SEQ ID NO: 119

SEQ ID NO: 123 - DNA, 683 bp, Epimedium_pubescens
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgccccttgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttattgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 124 - DNA, 683 bp, Epimedium_leptorrhizum
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggcctcccgtacctttgtaggcgcggccggcctaaaattcggccctcggcgacgagcgtcacgatctgtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 125 - DNA, 683 bp, Epimedium_simplicifolium
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgtggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 126 - DNA, 683 bp, Epimedium_pauciflorum
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcatgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 127 - DNA, 683 bp, Epimedium_rhizomatosum
gaacctgcggaaggatcattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcaagtctttgaacgcaagttgcgcccaaggccattaggtcgagggcacgtctgcctgggcgtcacgcacagcgtcgctcccaccatgatgcctttgttctgttatcgggcaactgcaacgtggcatgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 128 - DNA, 683 bp, Epimedium_brevicornum
gaacctgcggaaggtacattgtcaaaacctgcaaagcagaacgaccagcgaacttgtgaaaaacacttatgggagggacgaagggtgctaaccttgaatccttcctactgggtcacttgggacgattgtgtcgtgaaagcgacaacgacgcccctcgttgattcaaataacaactcggcgcggtctgcgccaaggaaaatcttaacggatagagcacgtcttcatgacgatgttgtaattccgatcttataacgactctcggcaatggatatctcggctctcgcatcgatgaagaacgtagcgaaatgcgatacttggtgtgaattgcagaatcccgtgaaccatcgagtctttgaacgcaagttgcgcccaaggccattaggtcgagggcacgtctgcctgggcgtcacgcacagcgtcgctcccaccatgatgcctttgttctgttatcgggcaactgcaacgtggcttgggaagcggatattggccccccgtacctttgtaggcgcggccggcctaaaattcggccctcggcgacgagcgtcacgatcagtggtggttgaataacccctttgtcatagaccggtatcgtgttgtttcgtcgtctatttgggccatatggacccttgcgtgtcgtataaacgacattcactctgcgaccccaggtcaggcgggactacccg

SEQ ID NO: 129 - DNA, 619 bp, Panax_ginseng
gtcgaaacctgcatagcagaacgacccgcgaacacgttacaataccgggtgagggacgaggggtgcgcaagctccccaagttgcaaacccatggtcggggaccacccttgggtggatctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagttgaggcggaggggcggataatggcctcccgtgtctcaccgcgcggttggcccaaatgcgagtccttggcgatggacgtcacgacaagtggtggttgtaaaaagccctcttctcatgtcgtgcggtgacccgtcgccagcaaaagctctcatgaccctgttgcgccgtcctcgacgtgcgctccgaccgcgacccc

SEQ ID NO: 130 - DNA, 619 bp, Panax_sinensis
gtcgaaacctgcatagcagaacgacccgcgaacacgttacaataccgggtgagggacgaggggtgcgcaagctccccaagttgcaaacccatggtcggggaccacccttgggtggctctcgtccgaacaacgaccccccggcgcggaatgcgccaaggaaatcaaactgaactgcacgcgtcccccccgtttgcgggcgatggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactccctcgcaggagttgaggcggaggggcggataatggcctcccgtgtctcaccgcgcggttggcccaaatgcgagtccttggcgatggacgtcacgacaagtggtggttgtaaaaagccctcttctcatgtcgtgcggttacccgtcgccagcaaaagctctcatgaccctgttgcgccgtcctcgacgtgcgctccgaccgcgacccc

SEQ ID NO: 131 - DNA, 619 bp, Panax_quinquefolius
gtcgaaacctgcatagcagaacgacccgcgaacacgttacaataccgggtgagggacgaggggtgcgcaagctccccaagttgcaaacccatggtcggggaccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcctttgcgggagtcgaggcggaggggcggataatggcctcccgtgtctcaccgcgcggttggcccaaatgcgagtccttggcgatggacgtcacgacaagtggtggttgtaaaaagccctcttctcatgtcgtgcggtgacccgtcgccagcaaaagctctcatgaccctgttgcgccgtcctcgacgtgcgctccgaccgcgacccc

SEQ ID NO: 132 - DNA, 619 bp, Panax_sinensis
gtcgaaacctgcatagcagaacgacccgcgaacacgttacaataccgggtgagggacgaggggtgcgcaagctccccaagttgcaaacccatggtcggggaccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactccctcgcaggagtcgaggcggaggggcggataatgccctcccgtgtctcaccgcgcggttggcccaaatgcgagtccttggcgatggacgtcacgacaagtggtggttgtaaaaagccctcttctcatgtcgtgcggtgacccgtcgccagcaaaagctctcatgaccctgttgcgccgtcctcgacgtgcgctccgaccgcgacccc

SEQ ID NO: 133 - DNA, 619 bp, Panax_pseudoginseng_var._elegantior
gtcgaaacctgcatagcagaacgacccgcgaacacgttacactatcgggtgagcgacgaggggtgcgcaagctccccaagttgcaaacccatggtcggggaccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggcggaggggcggataatggcctcccgtgtctcaccgcgcggttggcccaaatgcgagtccttggcgatggacgtcacgacaagtggtggttgtaaaaagccctcttcgcatgtcgtgcggtgacccgtcgccagcaaaagctctcatgaccctgttgcgccgtcctcgacgcgtgctccgaccgcgacccc

SEQ ID NO: 134 - DNA, 619 bp, Panax_variabilis
gtcgaaacctgcatagcagaacgacccgcgaacacgttacactaccgggtgagggacgaggggtgcgcaagctccccaagttgcaaacccatggtcggggaccacccttgggtggccctcgtccgaacaacgaccccccggcgcggaatgcgccaaggaaatcaaactgaactgtacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggtggaggggcggataatggcctcccgtgtctcaccgcgcggttggcccaaatacgagtccttggcgatggacgtcacgacaagtggtggttgtaaaaagccctcttctcatgtcgtgcggtgacccgtcgccagcaaaagctctcataaccctgttgcgccgtcctcgacgcgcgctccgaccgcgacccc

SEQ ID NO: 135 - DNA, 619 bp, Panax_variabilis
gtcgaaacctgcatagcagaacgacccgcgaacacgttacactaccgggtgagggacgaggggtgcgcaagctccccaagttgcaaacccatggtcggggaccacccttgggtggccctcgtccgaacaacgaccccccggcgcggaatgcgccaaggaaatcaaactgaactgtacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggtggaggggcggataatggcctcccgtgtctcaccgcgcggttggcccaaatacgagtccttggcgatggacgtcacgacaagtggtggttgtaaaaagccctcttctcatgtcgtgcggtgacccgtcgccagcaaaagctctcatgaccctgttgcgccgtcctcgacgcgcgctccgaccgcgacccc

SEQ ID NO: 136 - DNA, 623 bp, Panax_japonicus_var._bipinnatifidus
gtcgaaacctgcatagcagaacgacccgcgaacacgttacactatcgggtgagggacgaggggtgcgcaagctccccaagttgcaaacccatggtcggggaccacccttgggtggctctcgtccgaacaacgaccccccggcgcggaatgcgccaaggaaatcaaactgaactgcacgcgtcccccccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggcggaggggcggataatggcctcccgtgtctcaccgcgcggttggcccaaatgcgagtccttggcgatggacgtcacgacaagtggtggttgtaaaaagccctcttctcatgtcgtgcgctgacccgtcgccagcaaaagctctcatgaccctgttgcgccgtcctcgacgcgcgctccgaccgcgacccc

SEQ ID NO: 137 - DNA, 620 bp, Panax_pseudoginseng_var._bipinnatifidus
gtcgaaacctgcatagcagaacgacccgcgaacacgttacactaccgggtgagggacgaggggtgcgcaagctccccaagttgcaaacccatggtcggggtccacccttgggtggctctcgtccgaacaacgaccccccggcgcggaatgcgccaaggaaatcaaactgaactgcacgcgtccccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggcggaggggcggataatggcctcccgtgtctcaccgcgcggttggcccaaatgcgagtccttggcgatggacgtcacgacaagtggtggttgtaaaaagccctcttctcatgtcgtgcggtgacccgtcgccagcaaaagctctcatgaccctgttgcgccgtcctcgacgcgcgctccgaccgcgacccc

SEQ ID NO: 138 - DNA, 619 bp, Panax_wangianus
gtcgaaacctgcatagcagaacgacctgcgaacacgttacactaccgggtgagggatgaggggtgcgcaagctccccaagttgcaaacccatggtcggggacaacccttgggtggctctcgtccgaacaacgaacccccggcgcggaatgcgccaaggaaatcaaactgaactgcacgtgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtcgaggcggaggggcggataatggcctcccgtgtctcaccgcgcggttggcccaaatgcgagtccttggcgatggacgtcacgacaagtggtggttgtaaaaggccctcttctcatgtcgtgcggtgacctgtcgccagcaaaagctctcatgaccctgttgcgccgtcctcgacgcgcgctccgaccgcgacccc

SEQ ID NO: 139 - DNA, 618 bp, Panax_omeiensis
gtcgaaacctgcatagcagaacgacccgcgaacacgttacactaccgggtgagggacgaggggtgcgcaagctccccaagttgcaaacccatggtcggggtccacccttgggtggctctcgtccgaacaacgaccccccggcgcggaatgcgccaaggaaatcaaactgaactgacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtagaggcggaggggcggataatggcctcccgtgtctcaccgcgcggttggcccaaatgcgagtccttggcgatggacgtcacgacaagtggtggttgtaaaaagccctcttctcatgtcgtggggtgacccgtcgccagcaaaagctctcatgaccctgctgcgccgtcctcgacgcgcgctccgaccgcgacccc

SEQ ID NO: 140 - DNA, 619 bp, Panax_omeiensis
gtcgaaacctgcatagcagaacgacccgcgaacacgttacactaccgggtgagggacgaggggtgcgcaagctccccaagttgcaaacccatggtcggggtccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcgggaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacgcatcgcgtcgccccccaacccatcactcccttgcgggagtagaggcggaggggcggataatggcctcccgtgtctcaccgcgcggttggcccaaatgcgagtccttggcgatggacgtcacgacaagtggtggttgtaaaaagccctcttctcatgtcgtggggtgacccgtcgccagcaaaagctctcatgaccctgctgcgccgtcctcgacgcgcgctccgaccgcgacccc

SEQ ID NO: 141 - DNA, 619 bp, Panax_assamicus
gtcgaaacctgcatagcagaacgacccgcgaacacgttacactaccaggtgagggacgaggggtgcgcaagctccccaagtttcaaacccatggtcggggaccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgcagaatcccgtgaaccatcgagtctttgaacgcaagttgcgcccgaagccattaggccgagggcacgtctgcctgggcgtcacacatcgcgtcgccccccaacccatcactcccttgcgggagttgaggcggaggggcggataatggcctcccgtgtctcaccgcgcggttggcccaaatgcgagtccttggcgatggacgtcacgacaagtggtggttgtaaaaagccctcttctcatgtcgtgcggtgacccgtcgccagcaaaatctctcatgaccctgttgcgccgtcctcgacgcgcgctccgaccgcgacccc

SEQ ID NO: 142 - DNA, 618 bp, Panax_zingiberensis
gtcgaaacctgcatagcagaacgacccgcgaacacgttacactaccgggtgagggacgaggggtgcgcaagctccccaagttgcaaacccatggttggggaccacccttgggtggctctcgtccgaacaacgaccccccggcgcggaatgcgccaaggaaatcaaactgaactgcacgcgtcccccccgtttgcgggcggcggaagcgtctttctaaaacacaaacgactctcggcaacggatatctcggctctcgcatcgatgaagaacgtagcgaaatgcgatacttggtgtgaattgagaatcccgtgaaccatcgagtctttgaacgcaagttgcgcccgaagccattaggccgagggcacgtctgcctgggcgtcacgcatcgcgtcgccccccaacccatcactcccttgcgggagtcgaggcggaggggcggataatggcctcccgtgtctcaccgcgcggttggcccaaatgcgagtccttggcgatggacgtcacgacaagtggtggttgtaaaaagccctcttctcatgtcgtgcggtgacctgtcgccagcaaaagctctcatgaccctgttgcgccgtcctcgacgcgcgctccgaccgcgacccc

SEQ ID NO: 143 - DNA, 632 bp, Piper_methysticum
aaaacaagacagaccgaagtgaacttgtgagccctcgttattgacccgcacgcctgggcggcgctcggcacgcgtcgggcaaacaacgaaccaaatttcggcgcagcacgcgccaaggaaacatagatagctttgatcgcggcatcctctttccttgtcgggctggagggacgcatcgagcacttaccagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcttttgggtcgagggcacacctgcttgggcgttaaacaaatcgtcaccccgcaccttctctctccacgctccgagcaaggagtctggccgagttgagggtaggtcgggcggagttggccatccgtgtgcctgcagcgcacgtggttggcttaaaagctagggccatgggctgtgtgcggctcaacaagtggtggttgtgcgcccccagaaggccacgattgtcagcacgttgcgcctatcacttcttgaatgcgcccgcaaggggacccaacacaatcgaaacctctaatccacggatcgagagggaattgca

SEQ ID NO: 144 - DNA, 627 bp, Piper_cavendishioides
aaaacaagatagaccaaagagaactcgtaagccctcgttattggcccgcacgtctgggcgacgccctgcgcgcgtcgggcaacaacgaaccaaatctcggcgcagcacgcgccaaggaacttagaaagttttgatcgcagcctcctcctccccatcgggatggaggggacgcgtcgaacagttacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggccttccggtcgagggcacatctgcttgggcgttaaacaactcgtcaccccccaccttctctccccacactccgaccaaggagtggccggcggagacgagggcagggtgggcggagtctggttgtccgtgtgcctgcagcgcacgcggttggctgaaaagctagggccatgggctgtgtgcggctcaacaagtggtggttgtgcgccccgaaggccacgattgtcagtatgttgcgcccatcacttctcgatcgccctgcaaggacccaacacaatcgaaacatccaatcaacggatcgattcgaattgca

SEQ ID NO: 145 - DNA, 627 bp, Piper_colligatispicum
aaaacaagatagaccaaagagaactcgtaaaccctcgttattggcccgcacgtcggggcgacaccctgcgcgcgtcgggcaacaacgaaccaaatctcggcgcagcacgcgccaaggaacttagaaagttttgatcgcagcctcctcctccccatcgggatggaggggacgcatcgaacagttacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcctttcggtcgagggcacatctgcttgggcgttaaacaactcgtcgccccccaccttttctccccacactccgaccaaggagaggcaggcggagacgagggtagggtgggcggagtctggttgtccgtgtgcctgcagcgcacgcggttggctgaaaagctagggccatgggctgtgtgcggctcaacaagtggtggttgtgcgcctcgaaggccacgattgtcagcatgttgcgcccatcacttctcgattgccctgcaaggacccaacacaatcgaaacatccaatccacggatcgattcgaattgca

SEQ ID NO: 146 - DNA, 630 bp, Piper_capense
aaaacaagacagaccaaagcgaactcgtgaacccctcgttattggcccgcacgtcagggcggcgcccagcgcgtgtcgggcatcaacgaaccaaaactcggcgcagcacgcgccaaggaaattagaaagctttgattgcggcatcctcttccctatcgggctggagggacgcgtcgaacacttacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcctttcggtcgagggcacatctgcttgggcgttaaacaactcgtcgcccccccaccttctgcgtccacactccgagcaaggagtctggccgagatgaggggtagggttgggcggagtctggtcatccgtgtgcctgcagcgcatgcggtcggcttaaaagccagggccatgggctgtgtgcggctcaacaagtggtggttgtgcgccctcacaaggccacgattgtcagcacgttgcgccaatcacttctcgattgccctgcaaggacccaatacaatcgaaacatccaatccacggatcgattcgaattgca

SEQ ID NO: 147 - DNA, 627 bp, Piper_schuppii
aaaacaagatagaccaaagagaactcgtaagccctcgttattggcccgcacgtcggggcgacgccctgctcgcgtcgggcaacaacgaaccaaatctcggcgcagcacgcgccaaggaacttaaaaagttttgatcgcagcctcctccttcccatcgggatggaggggacgcgacgaacagttacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcctttcggtcgagggcacatctgcttgggcgttaaacaactcgtcgccccccaccttctctccccacactccgaccaaggagtggccggcggagacgagggtagggtgggcggagtctggctgtccgtgtgcctgcagcgcacgcggttggctgaaaagctagggccatgggctgtgtgcggctcaacaagtggtggttgtgcgcctcgaaggccacgattgtcagcatgttgcgcccatcacttctcgattgccttggaaggacccaacacaatcgaaacatccaatcaacggatcgattcgaattgca

SEQ ID NO: 148 - DNA, 626 bp, Piper_perpusillum
aaaacaaggtagaccaaagagaacttgtaaaccctcgttattggcccgcacgtcggggtgccgctctgcacgtgtcgggcaaacaacgaaccaaatctcggcgcaacacgcgccaaggaacttagaaagatttgattgcagcctcctcctccctattgggctggagggggcgcatcgaacactcacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcctttcggtcgagggcacatctgcttgggcgttaaacaactcgtcgccgcccaccttctctccccacactccgaccaaggagtatggcggagacgagggtagggcgggcggagtctggtcgtccgtgtgcctgcagtgcacgcggttggctgaaaaactagggccatgggctgtgtgcggctcaacaagtggtggttgtgcgccctgaaggccacgattgtcagcatgttgcacctatcacttctcaattgccctgcaaggacccagcgcaatcgaaacatccaatccacggatcgattcgaactgca

SEQ ID NO: 149 - DNA, 627 bp, Piper_trianae
aaaacaaggtagaccaaagagaacttgtaaaccctcgttattggcccgcacgtcggggtgccgctctgcacgtgtcgggcaaacaacgaaccaaatctcggcgcaacacgcgccaaggaacttagaaagatttgattgcagcctcctcctccctattgggctggagggggcgcatcgaacactcacaagtcaatacgactctcggcaacggatatctcggctctcgcatcgatgaagaacgtagcgaaatgcgatacttggtgtgaattgcagaatcccgtgaaccatcgagtctttgaacgcaagttgcgcccaaggcctttcggtcgagggcacatctgcttgggcgttaaacaactcgtcgccgcccaccttctctccccacactccgaccaaggagtatggcggaagacgagggtagggcgggcggagtctggtcgtccgtgtgcctgcagtgcacgcggttggctgaaaaactagggccatgggctgtgtgcggctcaacaagtggtggttgtgcgccctgaaggccacgattgtcagcatgttgcacctatcacttctcaattgccctgcaaggacccagcgcaatcgaaacatccaatccacggatcgattcgaactgca

SEQ ID NO: 150 - DNA, 1333 bp, Ginkgo_biloba
aagtgtcggatttaaagctggtgttaaagattacagattgacttattatactcctgaatatcaaaccaaagatactgatatcttggcagcattccgagtaactcctcaacctggagtgccacctgaggaagcgggagctgcagtagctgccgaatcttccactggtacatggaccactgtttggaccgatggacttaccagtcttgatcgttacaagggaagatgctatgacatcgagcccgttcctggggaggaaaatcaatttattgcctatgtagcttaccctttggatcttttcgaggaaggttctgttactaacctgttcacttccattgtagggaatgtatttggattcaaagccctacgagctctacgtctggaagatctgcgaattcctcctgcttattccaaaactttccagggtccacctcatggtatccaagttgaaagggataaattgaataaatatggccgtcccctattgggatgtactatcaagccaaaattgggtttatctgccaaaaattatggtagagcagtttacgaatgtcttcgtggtggacttgattttactaaagatgatgagaacgtaaattcccaaccattcatgcgctggagagatcgtttcttgttttgtgcagaagcaatttataaagctcagaccgagacgggtgaaattaagggacattacttgaatgctactgcaggtacatgcgaagaaatgatgaaaagggcagtatttgccagagaattgggagttcctatcgtcatgcatgactatctgacgggaggttttactgcaaatactagcttggctcattattgccgagacaatggcctacttcttcacattcaccgcgcaatgcatgcagttattgacagacaaagaaatcatggtatgcatttccgtgtactagctaaagcattgcgtatgtctggtggagatcatattcacgccggtactgtagtaggtaaacttgaaggagaacgagaagtcactctgggttttgttgatctactgcgtgatgattttattgaaaaagaccgaagtcgtggtatttatttcactcaagattgggtatctatgccaggtgttctgcccgtagcttcaggggatattcacgtctggcatatgcctgctctgaccgagatctttggggatgattctgtactacagtttggtgggggaactttgggacacccttggggaaatgcacctggtgcagtagctaatcgggtcgccctagaagcttgtgtacaagctcgtaatgaaggacgtgatcttgctcgtgaaggtaatgaagtgatccgtgaagctagtaaatggagtccc

SEQ ID NO: 151 - DNA, 1333 bp, Lepidozamia_hopei - G
aagtgtcggatttaaagctggtgttaaagattacagattaacttattacactcctgaatataaaaccaaagataccgatatcttggcagcgttccgagtaactccccaacctggggtgcccgctgaggaagcgggggctgcagtagctgctgaatcttccactggtacatggaccactgtttggaccgatggacttaccagtctcgatcgttacaaggggcgatgctatgacatcgagcccgttcctggggaagaaactcaatttattgcctatgtagcttaccccttagacctctttgaagaaggttctgttactaacatgttcacttccattgtaggtaatgtatttggattcaaagccctacgagctctacgtctagaagatttgcgaattcctcctgcttattccaaaactttccaaggtccacctcatggtatccaagttgaaagagataaattaaacaaatatggccgtcccctattgggatgtaccattaaaccaaaattggggttatctgccaaaaactatggtagagcagtttacgaatgtcttcgtggtggacttgattttaccaaagatgatgagaacgtaaattcccaaccatttatgcgctggagagatcgtttctgcttctgtgcagaagcaatttataaagctcaggctgagacgggtgaaattaagggacattacttgaatgctactgcgggtacatgtgaagaaatgataaaaagggcagtatttgccagagaattgggagttcctatcgtcatgcatgactacctgacgggaggttttactgcaaataccagcttggctcattattgccgagacaatggcctacttcttcacattcaccgcgcaatgcatgcagttattgacagacaaagaaatcatggtatgcacttccgtgtactagctaaagcattgcgcatgtccggtggagatcatgttcacgccggtactgtagtaggtaaacttgaaggagaacgagaagtcactctgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatctttggggatgattccgtactacagttcggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 152 - DNA, 1333 bp, Cycas_thouarsii - G
  misc_feature (545)..(545): n is a, c, g, or t
aagtgttggatttaaagctggtgttaaagattacagattaacttattacactcctgaatatcaaaccaaagataccgatatcttggcagcgttccgagtaactcctcaacctggagtgccgcctgaggaagcgggagctgcagtagccgctgaatcttccactggtacatggaccactgtttggaccgatggacttaccagtctcgatcgttacaaggggcgatgctatgacatcgagcccgttcctggggaggaaaatcaatttattgcctatgtagcttaccccttagacctctttgaagaaggttctgttactaacatgttcacttccattgtaggtaatgtatttggattcaaagccctacgagctctacgcctagaagatttgcgagttcctcctgcttattccaaaactttccaaggtccacctcatggtatccaagttgaaagagataaattaaacaaatatggccgtcctctattgggatgtactattaaacccaaattgggtttatctgccaaaaactatggtagagcagtttatnaatgtcttcgtggtggacttgattttaccaaagatgatgagaacgtaaattcccaaccatttatgcgctggagagatcgtttctgcttctgtgcagaagcaatttataaagctcaggctgagacgggtgaaattaagggacattacttgaatgctactgcaggtacatgcgaagaaatgatcaaaagggcagtatttgccagagaattgggagttcctatcgtcatgcatgactacctgacgggaggttttactgcaaataccagcttggctcattattgccgagacaatggcctacttcttcacattcaccgcgcaatgcatgcagttattgacagacaaagaaatcatggtatgcacttccgtgtgctagctaaagcattgcgaatgtccggtggagatcatattcacggcggtactgtagtaggtaaacttgaaggagaacgagacgtaactttgggttttgttgatctactgcgtgacgattttattgaaagagaccgaagtcgtggtatttatttcacccaagattgggtatctatgccaggtgttctgcccgtagcttcggggggtattcacgtttggcatatgcctgctctaaccgagatctttggggatgattccgtactacagttcggtgggggaactttgggacacccttggggaaatgcacctggtgcagtagcgaatcgagttgccttagaagcttgtgtacaagctcgtaatgaaggacgtgatcttgctcgtgaaggtaatgaagtgatccgcgaagctagtaaatggagtccc

SEQ ID NO: 153 - DNA, 1333 bp, Encephalartos_longifolius - G
  misc_feature (1179)..(1179): n is a, c, g, or t
aagtgtcggatttaaagctggtgttaaagattacagattaacttattacactcctgaatataaaaccaaagataccgatatcttggcagcgttccgagtaactccccaacctggggtgcccgctgaggaagcgggggctgcagtagctgctgaatcttccactggtacatggaccactgtttggaccgatggacttaccagtctcgatcgttacaaggggcgatgctatgacatcgagcccgttcctggggaagaaactcaatttattgcctatgtagcttaccccttagacctctttgaagaaggttctgttactaacatgttcacttccattgtaggtaatgtatttggattcaaagccctacgagctctacgtctagaagatttgcgaattcctcctgcttattccaaaactttccaaggtccacctcatggtatccaagttgaaagagataaattaaacaaatatggccgtcccctattgggatgtaccattaaaccaaaattgggtttatctgccaaaaactatggtagagcagtttacgaatgtcttcgtggtggacttgattttaccaaagatgatgagaacgtaaattcccaaccatttatgcgctggagagatcgtttctgcttttgtgcagaagcaatttataaagctcaggctgagacgggtgaaattaagggacattacttgaatgctactgcaggtacatgtgaagaaatgataaaaagggcagtatttgccagagaattgggagttcctatcgtcatgcatgactacctgacgggaggttttactgcaaataccagcttggctcattattgccgagacaatggcctacttcttcacattcaccgcgcaatgcatgcagttattgacagacaaagaaatcatggtatgcacttccgtgtactagctaaagcattgcgcatgtccggtggagatcatattcacgccggtactgtagtaggtaaacttgaaggagaacgagaagtcactctgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatctttggggatgattccgtactacagtncggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 154 - DNA, 1333 bp, Encephalartos_munchii - G
aagtgtcggatttaaagctggtgttaaagattacagattaacttattacactcctgaatataaaaccaaagataccgatatcttggcagcgttccgagtaactccccaacctggggtgcccgctgaggaagcgggggctgcagtagctgctgaatcttccactggtacatggaccactgtttggaccgatggacttaccagtctcgatcgttacaaggggcgatgctatgacatcgagcccgttcctggggaagaaactcaatttattgcctatgtagcttaccccttagacctctttgaagaaggttctgttactaacatgttcacttccattgtaggtaatgtatttggattcaaagccctacgagctctacgtctagaagatttgcgaattcctcctgcttattccaaaactttccaaggtccacctcatggtatccaagttgaaagagataaattaaacaaatatggccgtcccctattgggatgtaccattaaaccaaaattgggtttatctgccaaaaactatggtagagcagtttacgaatgtcttcgtggtggacttgattttaccaaagatgatgagaacgtaaattcccaaccatttatgcgctggagagatcgtttctgcttttgtgcagaagcaatttataaagctcaggctgagacgggtgaaattaagggacattacttgaatgctactgcaggtacatgtgaagaaatgataaaaagggcagtatttgccagagaattgggagttcctatcgtcatgcatgactacctgacgggaggttttactgcaaataccagcttggctcattattgccgagacaatggcctacttcttcacattcaccgcgcaatgcatgcagttattgacagacaaagaaatcatggtatgcacttccgtgtactagctaaagcattgcgcatgtccggtggagatcatattcacgccggtactgtagtaggtaaacttgaaggagaacgagaagtcactctgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatctttggggatgattccgtactacagttcggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 155 - DNA, 1333 bp, Encephalartos_trispinosus - G; identical to SEQ ID NO: 154

SEQ ID NO: 156 - DNA, 1333 bp, Ceratozamia_kuesteriana - G
aagtgtcggatttaaagctggtgttaaagattacagattaacttattacactcctgaatataaaaccaaagataccgatattttggcagcgttccgagtaactccccaacctggggtgccacctgaggaagcgggggctgcagtagctgctgaatcttccactggtacatggaccactgtttggaccgatggacttaccagtctcgatcgttacaaggggcgatgctatgacatcgagcccgttcctggggaagagactcaatttattgcctatgtagcttaccccttagacctctttgaagaaggttctgttactaacatgttcacttccattgtaggtaatgtatttggattcaaagccctacgagctctacgtctagaagatttgcgagttcctcctgcttattccaaaactttccaaggtccacctcatggtatccaagttgaaagagataaattaaacaaatatggacgtcccctattgggatgtaccattaaaccaaaattgggtttatctgccaaaaactatggtagagcagtttacgaatgtcttcgtggtggacttgattttaccaaagatgatgagaacgtaaattcccaaccatttatgcgctggagagatcgtttctgcttctgtgcagaagcaatttataaagctcaggctgagacgggtgaaattaagggacattacttgaatgctactgcaggtacatgtgaagaaatgataaaaagggcagtatttgccagagaattgggagttcctatcgtcatgcatgactacctgacgggaggttttactgcaaataccagcttggctcattattgtcgagacaatggcttacttcttcacattcaccgcgcaatgcatgcagttattgacagacaaagaaatcatggtatgcacttccgtgtactagctaaagcattgcgcatgtccggtggagatcatattcacgccggtactgtagtaggtaaacttgaaggagaacgagaagtcactttgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatctttggggatgattccgtactacagttcggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 157 - DNA, 1333 bp, Macrozamia_lucida - G
aagtgtcggatttaaagctggtgttaaagattacagattaacttattacactcctgaatataaaaccaaagataccgatatcttggcagcgttccgagtaactccccaacctggggtgcccgctgaggaagcgggggctgcagtagctgctgaatcttccactggtacatggaccactgtttggaccgatggacttaccagtctcgatcgttacaaggggcgatgctatgacatcgagcccgttcctggggaagaaactcaatttattgcctatgtagcttaccccttagacctctttgaagaaggttctgttactaacatgttcacttccattgtaggtaatgtatttggattcaaagccctacgagctctacgtctagaagatttgcgaattcctcctgcttattccaaaactttccaaggtccacctcatggtatccaggttgaaagagataaattaaacaaatatggccgtcccctattgggatgtaccattaaaccaaaattgggtttatctgccaaaaactatggtagagcagtttacgaatgtcttcgtggtggacttgattttaccaaagatgatgagaacgtaaattcccaaccatttatgcgctggagagatcgtttctgcttctgtgcagaagcaatttataaagctcaggctgagacgggtgaaattaagggacattacttgaatgctactgcaggtacatgtgaagaaatgataaaaagggcagtatttgccagagaattgggagttcctatcgtcatgcatgactacctgacgggaggttttactgcaaataccagcttggctcattattgccgagacaatggcctacttcttcacattcaccgcgcaatgcatgcagttattgacagacaaagaaatcatggtatgcacttccgtgtactagctaaagcattgcgcatgtccggtggagatcatattcacgccggcactgtagtaggtaaacttgaaggagaacgagaagtcactttgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatatttggggatgattccgtactacagttcggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 158 - DNA, 1333 bp, Ceratozamia_norstogii - G
aagtgtcggatttaaagctggtgttaaagattacagattaacttattactctcctgaatataaaaccaaagataccgatattttggcagcgttccgagtaactccccaacctggggtgccacctgaggaagcgggggctgcagtagctgctgaatcttccactggtacatggaccactgtttggaccgatggacttaccagtctcgatcgttacaaggggcgatgctatgacatcgagcccgttcctggggagagagatcaatttattgcctatgtagcttaccccttagacctctttgaagaaggttctgttactaacatgttcacttccattgtaggtaatgtatttggattcaaagccctacgagctctacgtctagaagatttgcgagttcctcctgcttattccaaaactttccaaggtccacctcatggtatccaagttgaaagagataaattaaacaaatatggacgtcccctattgggatgtaccattaaaccaaaattgggtttatctgccaaaaactatggtagagcagtttacgaatgtcttcgtggtggacttgattttaccaaagatgatgagaacgtaaattcccaaccatttatgcgctggagagatcgtttctgcttctgtgcagaagcaatttataaagctcaggctgagacgggtgaaattaagggacattacttgaatgctactgcaggtacatgtgaagaaatgataaaaagggcagtatttgccagagaattgggagttcctatcgtcatgcatgactacctgacgggaggttttactgcaaataccagcttggctcattattgtcgagacaatggcttacttcttcacattcaccgcgcaatgcatgcagttattgacagacaaagaaatcatggtatgcacttccgtgtactagctaaagcattgcgcatgtccggtggagatcatattcacgccggtactgtagtaggtaaacttgaaggagaacgagaagtcactttgggttttgttgatctactgcgtgatgattttattgaaagagaccgaagtcgtggtatttatttcactcaagattgggtatctatgccaggtgttctgcccgtagcttcggggggtattcacgtttggcatatgcctgctctgaccgagatctttggggatgattccgtactacagttcggcgggggaactttgggacacccttggggaaatgcacctggtgcagtagcgaatcgagtcgccttagaagcttgtgtacaagctcgtaatgagggacgtgatcttgctcgtgaaggtaatgaagtgatccgtgaagctagtaaatggagtccc

SEQ ID NO: 159 - DNA, 1413 bp, Hydrastis_canadensis
  misc_feature (41)..(41): n is a, c, g, or t
  misc_feature (434)..(434): n is a, c, g, or t
ctcaaatcattggtccggtactagatgtagcctttccagcnggcaagatgcctaatatttacaacgctttggtagttaaaggtcaagatacagctggccggcaaattaatgtaacttgtgaggtgcagcaattattaggaaataaccgagttagagcggtagctatgagtgctacagagggtctgatgagaggaatggaagtgatggacacgggagctcctctaagtgttccagttggtggagctactctcggacgaattttcaacgttcttggagagcctgttgataattttggtcctgtagatactcgcacaacatctcctattcatagatctgcgcctgcctttatacagttagataccaaattatctatttttgaaacggggattaaagtagtggatcttttagctccttatcgccgtggaggaaaaatnggactatttgggggagctggagtgggtaaaacagtactcatcatggaattgatcaacaacattgcgaaagctcatggaggtgtatccgtatttggcggagtaggcgaacgtactcgtgaaggaaatgatctttacatggaaatgaaagaatcaggagtgattaatgaacaaaatcttgcagaatcaaaagtcgcgctagtctatggtcagatgaatgaaccgccgggagctcgtatgagagttggtttgactgccctaaccatggcagaatatttccgggatgttaatgagcaagacgtacttctatttatcgacaatatttttcgtttcgtccaagcaggatccgaagtatccgctttattaggcagaatgccttctgctgtaggttatcaacctactcttagtacagaaatgggttctttgcaagaaagaattacttctaccaaagagggatccataacttctattcaagcagtttatgtacctgcggacgatttgaccgaccccgctcctgccacaacatttgcgcatttagatgctactaccgtactatcaagaggactggcttcaaaaggtatctatccagcagtagatccgttagattcaacgtcaactatgctacaacctcggatcgttggtgaggaacattatgaaactgcgcaaagagttaagcaaacttcacaacgttacaaagaacttcaggacattatagctatccttgggttggacgaattatccgaagaggatcgtttaaccgtagcaagagcacgaaaaattgagcgtttcttatcacaacccttcttcgtagcagaagtatttaccggttctccaggaaaatatgtcggtctaacagaaacaattagggggtttcaactgatcctttccggagaattagatggtcttccggagcaggccttttatttggtgggtaacatcgacgaagctaccgcgaaggctatgaacttagatgtggagagca

SEQ ID NO: 160 - DNA, 1413 bp, Stauntonia_hexaphylla - HYD
  misc_feature (1384)..(1386): n is a, c, g, or t
ctcaactcattggtccggtactggatgtagcctttccaccgggcaagatgcctaatatttacaacgctttggtagttaaaggtcgagatactgttgaccagcaaattaatgtgacttgtgaggtacagcaattattaggaaataatcgagttagagctgtagctatgagtgctacagatggtctgatgagaggaatgaaagtgattgacacgggagctccactaagtgttccagtcggtggagcgactcttggacgaattttcaatgttcttggagagcctgttgataatttaggtcctgtagatactcgcacaacatctcctattcatagatctgcgcctgcctttatacagttagatacaaaattatctatttttgaaacagggattaaagtagtggatcttttagctccttatcgccgtggaggaaaaattggactattcggaggagctggagtgggtaaaacagtactcatcatggaattgatcaacaacattgccaaagctcatgggggtgtatctgtatttggcggagtaggtgaacgtactcgtgaaggaaatgatctttacatggaaatgaaagaatctggagtaattaatgaacaaaatattccagaatctaaagtagctctagtttatggtcagatgaatgaaccgccgggagctcgtatgagagttggtttgactgctctaaccatggcggaatatttccgggatgttaatgaacaagacgtacttctttttatcgacaatatttttcgtttcgtccaagcaggatccgaagtatctgctttattaggcagaatgccttctgctgtaggttatcaacctacccttagtacagaaatgggttctttgcaagaaagaattacttctaccaaagagggatccataacttctattcaagcagtttatgtacctgcggacgatttgaccgaccctgctcctgccacgacatttgcacatttagatgctactaccgtactatcaagaggactagctgccaaaggtatctatccagcagtagatcctttagattcaacgtcaactatgctccaacctcggatcgttggtgaggaacattatgaaactgcgcaaagagttaagcaaacttcacaacgttacaaagaacttcaggacattatagctatccttggattggacgaattatccgaagatgatcgtttaaccgtagcaagagcacgaaaaattgagcgtttcttatcacaacccttcttcgtagcagaagtatttactggttctccagggaaatatgttggtttaacagaaacaattagggggtttcaactgatcctttccggagaattagatggtcttcctgagcaggccttttatttggtgggtaacatcgatgaagctaccgnnnaggctatgaacttagaagtggagagca

SEQ ID NO: 161 - DNA, 1413 bp, Cocculus_trilobus - HYD
cgcaaatcattggtccggtactggatgtagtctttccaccaggcaagatgcctaatatttacaacgctttggtagtgaaaggtcaagatactgttggccagcaaattaatgtaacttgtgaagtacagcaattattaggaaataatcgagttagagctgtagctatgagtgcgacggatggtctgatgagaggaatggaagtgattgacacaggagctcctctaagtgttccagtcggtggagcaactctcggacgcattttcaacgttcttggagaacctgttgataatttgggtcctgtagatagtcgtacaacatctcctattcataggtctgcgcctgcctttatacagttagatacaaaattatctatttttgaaacaggaattaaagtagtggatcttttagctccttatcgccgtggaggaaaaatcggactattcgggggagctggggtgggtaaaacagtgctcatcatggaattgatcaacaacattgccaaagctcatggcggcgtatccgtatttggcggagttggcgaacgtactcgtgaaggaaatgatctttacatggaaatgaaagaatctggagtgattaatgaacaaaatattgtagaatcaaaagtagcactagtctatggtcagatgaatgaaccgccgggagctcggatgagagttggtttgactgccctaaccatggcggaatatttccgggatgttaatgaacaagacgtacttctatttatcgacaatatttttcgtttcgtccaagccggatccgaagtctccgctttattaggcagaatgccttctgctgtaggttatcaacctactcttagtacagaaatgggttctttgcaagaaagaattacttctaccaaagagggatccataacttctattcaagcagtttatgtacctgcggacgatttgaccgaccccgcccctgccacgacatttgcgcatttagatgctactaccgtactatcaagaggactagctgccaaaggtatctatccagcagtagatcctttagattcaacgtcaactatgctccaacctcggattgttggcaaggaacattatgaaactgcgcaaagagttaagcaaacttcacaacgttacaaagaacttcaagacattatagctatccttgggttggacgaattatccgaagaggatcgtttaaccgtagcaagagcgcgcaaaattgagcgtttcttatcacaacccttcttcgtagcagaagtatttaccggttctccaggaaaatatgttggtctaacagaaacaattagggggtttcaactgatcctttccggagaattagatggtcttcctgagcaggccttttatttggtgggtaacatcgatgaagctaccgcgaaggctatgaacttagatgaggagagca

SEQ ID NO: 162 - DNA, 1413 bp, Euptelea_polyandra - HYD
  misc_feature (802)..(802): n is a, c, g, or t
  misc_feature (806)..(806): n is a, c, g, or t
ctcaaatcattggtccggtactggatgtagcctttcccccgggcaagatgcctaatatttataacgctttggtagttaagggtcgagatactgtcggccagcaaattaatgtgacttgtgaggtacaacaattattaggaaataatcgagttagagctgtagctatgagtgctacagatggtctgatgagaggaatggaagtgattgacacgggagctcctctaagtgttccagtcggtggagcgactctcggacgaattttcaacgttcttggagagcctgttgataatttaggtgctgtagatactcgcacaacatctcctattcatagatctgcgcctgcctttatacagttagataccaaattatctatttttgaaacagggattaaagtagtggatcttttagctccttatcgccgtggaggaaaaatcggactattcgggggagctggagtgggtaaaacagtactcatcatggaattgatcaacaacattgccaaagctcatggaggcgtatctgtatttggcggagtaggcgaacgtactcgtgaaggaaatgatctttacatggaaatgaaagaatctggagtgattaatgaacaaaatattgcagaatcaaaagtagctctagtctatggtcagatgaatgaaccgccgggagctcgtatgagagttggtttgactgccctaaccatggcggaatattttcgggatgttaatgaacaagacgtacttctatttatcgacaatattttccgtttcgtccaagcaggatctgaagtatctgccttattaggcanaatnccttctgctgtgggttatcaacctacccttagtacagaaatgggttctttgcaagaaagaattacttccaccaaagagggatccataacttctattcaagcagtttatgtacctgcggacgatttgaccgaccctgctcctgccacgacatttgcgcatttagatgctactaccgtactatcaagaggactagctgccaaaggtatctatccagcagtagatcctttagattcaacgtcaactatgctccaaccttggatcgttggtgaggaacattatgaaactgcgcaaagagttaagcaaacttcacaacgttacaaagaacttcaggacattatagctatccttgggttggacgaattatccgaagaggatcgtttaaccgtagcaagagcacgaaaaattgagcgtttcttatcccaacccttcttcgtagcagaagtatttaccggttctccagggaaatatgttggtctagcagaaacaattagggggtttcaactgatcctttccggagaattagatggtcttcccgaacaggccttttatttggtaggtaacatcgatgaagctaccgcgaaggctatgaacttagaagtggagagca

SEQ ID NO: 163 - DNA, 1413 bp, Euptelea_polyandra_2 - HYD; identical to SEQ ID NO: 162
  misc_feature (802)..(802): n is a, c, g, or t
  misc_feature (806)..(806): n is a, c, g, or t

SEQ ID NO: 164 - DNA, 1413 bp, Sargentodoxa_cuneata - HYD
  misc_feature (390)..(390): n is a, c, g, or t
  misc_feature (701)..(701): n is a, c, g, or t
  misc_feature (1268)..(1268): n is a, c, g, or t
  misc_feature (1320)..(1320): n is a, c, g, or t
  misc_feature (1373)..(1373): n is a, c, g, or t
ctcaaatcattggtccggtactagatgtagcctttccaccgggcaagatgcctaatatttacaacgctttggtagttaaaggtcgagatactgttgaccagcaaattaatgtgacttgtgaggtacagcaattattaggaaataatcgagttagagctgtagctatgagtgctacggatggtctgatgagaggaatgaaagtgattgacacgggagctccactaagtgttccagtcggtggagctactcttggacgaattttcaacgttcttggagagcctgttgataatttaggtcctgtagatactcgcacaacatctcctattcatagatctgcgcctgcctttatacagttagatacaaaattatctatttttgaaacaggaattnaagtagtggatcttttagccccttatcgccgtggaggaaaaattggactattcgggggagctggagtgggtaaaacagtactcatcatggaattgatcaacaacattgccaaagctcatgggggtgtatccgtatttggcggagtaggtgaacgtactcgtgaaggaaatgatctttacatggaaatgaaagaatctggagtgattaatgaacaaaatattccagaatccaaagtagctctggtttatggtcagatgaatgaaccgccgggagctcgtatgagagttggtttgactgctctaaccatggcngaatatttccgggatgttaatgaacaagacgtacttctatttatcgacaatatttttcgtttcgtccaagcaggatccgaagtatctgctttattaggcagaatgccttctgctgtaggttatcaacccacccttagtacagaaatgggttctttgcaagaaagaattacttctaccaaagagggatccataacttctattcaagcagtttatgtacctgcggacgatttgaccgaccctgctcctgccacgacatttgcacatttagatgctactaccgtactatcaagaggactagctgccaaaggtatctatccagcagtggatcctttagattcaacgtcaacgatgctccaacctcggatcgttggtgaggaacattatgaaactgcgcaaagagttaagcaaacttcacaacgttacaaagaacttcaggacattatagctatccttgggttggacgaattatccgaagacgatcgtttaaccgtagcaagagcacgaaaaatcgagcgtttcttatcacaacccttcttcgtagcagaagtatttaccggttctccaggnaaatatgttggtctaacagaaacaattaggggatttcaactgatcctttccngagaattagatgggcttcctgagcaggccttttatttggtgggtaacatcgangaagctaccgcgaaggctatgaacttagaagtggagagca

SEQ ID NO: 165 - DNA, 1413 bp, Menispermum_canadense - HYD
  misc_feature (173)..(173): n is a, c, g, or t
  misc_feature (801)..(801): n is a, c, g, or t
ctcaaatcattggtccggtactggatgtagtctttccaccaggcaagatgcctaatatttacaacgctttggtagtgaaaggtcaagatactgtcggccagcaaattaatgtaacttgtgaggtacagcaattactaggaaataatcgagttagagctgtagctatgagtgcnacggatggtctgatgagaggaatggaagtgattaacacgggagctcctctaagtgttccagtcggtggagcaactctcggacgcattttcaacgttcttggagaacctgttgataatttaggtcctgtagatagtagcacaacatctcctattcataggtctgcgcctgcctttatacagttagatacaaaattatctatttttgaaacaggaattaaagtagtggatcttttagctccttatcgccgtggaggaaaaatcgggctattcgggggagctggagtgggtaaaacagtgctcatcatggaattgatcaacaacattgccaaagctcatggcggcgtatccgtatttggcggagttggcgaacgtactcgtgaaggaaatgatctttacatggaaatgaaagaatctggagtgattaatgaacaaaatattgtagaatccaaagtagcgctagtctatggtcagatgaatgaaccgccgggagctcggatgagagttggtttgactgccctaaccatggcggaatatttccgggatgttaatgaacaagacgtacttctatttatcgacaatatttttcgtttcgtccaagcaggatccgaagtctccgctttattaggcngaatgccttctgctgtaggttatcaacctactcttagtacagaaatgggttctttgcaagaaagaattacttctaccaaagagggatccataacttctattcaagcagtttatgtacctgcggacgatttgaccgaccccgctcctgccacgacatttgcacatttagatgctactaccgtactatcaagaggactagctgccaagggtatctatccagcagtagatcctttagattcaacgtcaactatgctccaacctcggattgttggtaaggaacattatgaaactgcgcaaagagttaagcaaacttcacaacgttacaaagaacttcaagacattatagctatccttgggttggacgaattatccgaagaggatcgtttaaccgtagcaagagcacgaaaaattgagcgtttcttatcacaacccttcttcgtagcagaagtatttaccggttctccaggaaaatatgttggtctaacagaaacaattagggggtttcaactgatcctttccggagaattagatggtcttcctgagcaggccttttatttggtgggtaacatcgatgaagctaccgcgaaggctatgaacttagacggggagagca

SEQ ID NO: 166 - DNA, 1413 bp, Coptis_trifolia - HYD
  misc_feature (773)..(773): n is a, c, g, or t
ctcaaatcattggtccggtactggatgtatcctttccggcgggcaagatgcctaatatttacaatgctttggtagttaaaggtcaagatactgttggccagcaaattaacgtaacttgcgaggtacagcaattattaggaaataatcgagttagggctgtagctatgagtgcgacagacggtctgatgagaggaatggaagtgattgacacgggagctcctctgagtgttccagtcggtggagcgactctcggacgaattttcaatgttcttggagaacctgttgataatttgggtcctgtagatactcgcacaacatctcctattcatagatctgcgcctgcctttatacaattagatactaaattatctatttttgaaacggggattaaagtagtggatcttttagctccttatcgccgtggaggaaaaatcgggctattcgggggggctggagtgggtaaaacagtactcatcatggaattgatcaacaacattgccaaagctcatggaggtgtatccgtatttggcggagtgggcgaacgtactcgcgaaggaaatgatctttacatggaaatgaaagaatccggagtgattaatgaacaaaatatttcggaatcaaaagtagctctagtctatggtcagatgaatgaaccgccgggagctcgtatgagagttggtttgactgccctaaccatggcggaatatttccgggatgttaatgaacaagacgtgcttctatttatcgacaatatttttcgtttcgtccaagcnggatccgaagtatccgctttattaggaagaatgccttccgctgtaggttatcaacctactcttagtacagaaatgggttctttgcaagaaagaattacttctacgaaagagggatccataacttcgattcaagcagtttatgtacctgcagacgatttgaccgaccccgctcctgccacaacatttgcgcatttagatgctactaccgtactatcaagagggctggctgccaaaggtatctatccagcagtagatcctttagattcaacgtcaactatgctccaacctcggatcgttggtgaggaacattatgaaactgcgcaaagagttaagcaaacttcacaacgttacaaagaacttcaggacattatagctatccttgggttggacgaattatccgaagaggatcgtttaaccgtagcaagagcacgaaaaattgagcgtttcttatcacaacccttcttcgtagcagaagtttttaccggttctcccggaaaatatgttggtctaacagaaacaattagggggtttcaactgattctttccggagaattagacggtcttccggagcaggccttttatttggtaggtaatatcgatgaagctaccgcgaaggctatgaacttagatgtggagagca

SEQ ID NO: 167 - DNA, 1413 bp, Xanthoriza_simplicissima - HYD
  misc_feature (392)..(392): n is a, c, g, or t
  misc_feature (1385)..(1385): n is a, c, g, or t
ctcaaatcattggtccggtactggatgtatcctttccggcgggcaagatgcctaatatttacaacgctttggtagttaaaggtcaagatactgttggccagcaaattaacgtaacttgcgaggtacagcaattattaggaaataatcgagttagggctgtagctatgagtgcgacagacggtcttatgagaggaatggaagtgattgacacgggagctcctctgagtgttccagtcggtggagcgactctcggacgaattttcaatgttcttggagaacctgttgataatttgggtcctgtagatactcgcacaacatctcctattcatagatctgcgcctgcctttatacagttagataccaaattatctatttttgaaacggggattaangtagtggatcttttagctccttatcgccgtggaggaaaaatcgggctattcgggggggctggagtgggtaaaacagtactcatcatggaattgatcaacaacattgccaaagctcatggaggtgtatccgtatttggcggagtgggcgaacgtactcgcgaaggaaatgatctttacatggaaatgaaagaatccggagtgattaatgaacaaaatattgcggaatcaaaagtagctctagtctatggtcagatgaatgaaccgccgggagctcgtatgagagttggtttgactgccctaaccatggcggaatatttccgggatgttaatgaacaagacgtgcttctatttatcgacaatatttttcgtttcgtccaagcgggatctgaagtatccgctttattaggaagaatgccttccgctgtaggttatcaacctactcttagtacagaaatgggttctttgcaagaaagaattacttctacgaaagagggatccataacttcgattcaagcagtttatgtacctgcagacgatttgaccgaccccgctcctgccacaacatttgcgcatttagatgctactaccgtactatcaagagggctggctgccaaaggtatctatccagcagtagatcctttagattcaacgtcaactatgctccaacctcggatcgttggtgaggaacattatgaaactgcgcaaagagttaagcaaacttcacaacgttacaaagaacttcaggacattatagctatccttgggttggacgaattatccgaagaggatcgtttaaccgtagcaagagcacgaaaaattgagcgtttcttatcacaacccttcttcgtagcagaagtttttaccggttctcccggaaaatatgttggtctaacagaaacaattagggggtttcaactgattctttccggagaattagacggtcttccggagcaggccttttatttggtaggtaatatcgatgaagctaccgcnaaggctatgaacttagatgtggagagca

SEQ ID NO: 168 - DNA, 1413 bp, Sinofranchetia_chinensis - HYD
  misc_feature (183)..(183): n is a, c, g, or t
  misc_feature (185)..(185): n is a, c, g, or t
  misc_feature (805)..(809): n is a, c, g, or t
ctcaaatcattggtccggtactggatgtagcctttccgccgggcaagatgcctaatatttacaacgctttggtagttaaaggtcgagatactgttgaccagcaaattgatgtgacttgtgaggtacagcaattattaggaaataatcgagttagagctgtagctatgagtgctacagatggtntnatgagaggaatgaaagtgattgacacgggagctccactaagtgttccagtcggtggagcgactcttggacgaattttcaacgttcttggagagcctgttgataatttaggtcctgtagatactcgcacaatatctcctattcatagatctgcgcctgcctttatacagttagatacaaaattatctatttttgaaacagggattaaagtagtggatcttttagctccttatcgccgtggaggaaaaattggactattcggaggagctggagtgggtaaaacagtactcatcatggaattgatcaacaacattgccaaagctcatggaggtgtatctgtatttggcggagtaggtgaacgtactcgtgaaggaaatgatctttacatggaaatgaaagaatctggagtgattaatgaacaaaatattccagaatctaaagtagctctagtttatggtcagatgaatgaaccgccgggagctcgtatgagagttggtttgactgctctaaccatggcggaatatttccgggatgttaatgaacaagacgtacttctatttattgacaatatttttcgtttcgtccaagcaggatccgaagtatctgctttattaggcagaannnnntctgctgtaggttatcaacctacccttagtacagaaatgggttctttgcaagaaagaattacttctaccaaagagggatccataacttctattcaagcagtttatgtacctgcggacgatttgaccgaccccgctcctgccacgacatttgcacatttagatgctactaccgtactatcaagaggactagctgccaaaggtatctatccagcagtagatcctttagattcaacgtcaactatgctccaacctcggatcgttggtgaggaacattatgaaactgcgcaaagagttaagcaaacttcacaacgttacaaagaacttcaggacattatagctatccttgggttggacgaattatccgaagaggatcgtttaaccgtagcaagagcacgaaaaattgagcgtttcttatcacaacccttcttcgtagcagaagtatttactggttctccagggaaatatgttggtctaacagaaacaattagggggtttcaactgatcctttccggagaattagatggtcttcctgagcaggccttttatttggtgggtaacatcgatgaagctaccgcgaaggctatgaacttagaagtggagagca

SEQ ID NO: 169 - DNA, 609 bp, Pausinystalia_johimbe
tcgaatcctgcgaaacgcacgaccgtgaacctgtgtcaacagccgggcgtcgggtggcaggggagaataacccctcccttccataccggcgccttccgggtgcccgtcgcgcggaaaatgtaactcaaacccggcgcggaacgcgccaaggaaaactcaataggattgtcaaaccccccgatgccccgttcgcggtgtgctagggggtgttgtggcgcctgtcgtaatccaaacgactctcggcaacggatatctcggctctcgcatcgatgaagaacgtagcgaaatgcgatacttggtgtgaattgcagaatcccgcgaaccatcgagtctttgaacgcaagttgcgcccgaagccgtcaggctgagggcacgtctgcctgggcgtcacgcatcgcgtcgctgcccccacaagtcgtgtggggcggcggatgttggcctcccgtgctgtcaagcgtggccggcctaaatgagagtcctcggcgagggacgtcacgatgagtggtggttgaatgccccgactcgatttctgtcgtgccggttcccctcgctgtctctggctccacgaatgaccctcgcgcgctctctctttggagtcgcgctccgacc

SEQ ID NO: 170 - DNA, 609 bp, Pausinystalia lane-poolei subsp. lane-poolei
tcgaatcctgcgaaacgcacgaccgtgaacctgtgtcaacagccgggcgtcgggtggcaggggagactaacccctcccttccatcccggcgccctccgcgtgcctgtcgcgcggaaaacgtaactcaaacccggcgcggaacgcgccaaggaaaactcaataggattgtcaaaccccccgatgccccgttcgcggtgtgctagggggtgttgtggcgcctgtcgtaatccaaacgactctcggcaacggatatctcggctctcgcatcgatgaagaacgtagcgaaatgcgatacttggtgtgaattgcagaatcccgcgaaccatcgagtctttgaacgcaagttgcgcccgaagccgtcaggccgagggcacgtctgcctgggcgtcacgcatcgcgtcgctgcccccacaagtcgtgtggggcggcggatgttggcctcccgtgccgtcaagcgcggccggcctaaatgagagtcctcggcgagggacgtcacgacgagtggtggttgaatgccccgactcgatttctgtcgtgccggttccccttgctctctccggctccacgaatgaccctcgcgcgctctctatttggagtcgcgctccgacc

SEQ ID NO: 171 - DNA, 609 bp, Pausinystalia_talbotii
tcgaatcctgcgaaacgcacgaccgtgaaacttgtgtcaacagccgggcgtcgggtggcaggggagactaacccctcccttccatcccggcgccctccgcgtgcccgtcgcgcggaaaatgtaactcaaacccggcgcggaacgcgccaaggaaaactcaataggattgtcaaaccccccgatgccccgttcgcggtgtgctaaggggggttgtggcacctgtcgtaatccaaacgactctcggcaacggatatctcggctctcgcatcgatgaagaacgtagcgaaatgcgatacttggtgtgaattgcagaatcccgcgaaccatcgagtctttgaacgcaagttgcgcccgaagccgtcaggccgagggcacgtctgcctgggcgtcacgcatcgcgtcgctcccccacaagtcgtgtggggcagcggatgttggcctcccgtgccgtcaagcgcggccggcctaaatgagagtcctcggcgagggacgtcacgacgagtggtggttgaatgccccgactcgatttctgtcgtgccggttcccctcgctgtctccggctctacgaatgaccctcgcgcgctctctatttggagtcgcgctccgacc

SEQ ID NO: 172 - DNA, 610 bp, Corynanthe_paniculata - PAU
tcgaatcctgcgaaacgcacgaccgtgaacgtgtgtcaacagccgggcgtcgggtggcaggggagactaacccctcccttccatgcccggcgccctccgcgtgcccgtcgcgcggaaaatgtaactcaaacccggcgcggaacgcgccaaggaaaactcaataggattgtcaaaccccccgatgccccgttcgcggtgtgctagggggcgttgtggcacctgtcgtaatccaaacgactctcggcaacggatatctcggctctcgcatcgatgaagaacgtagcgaaatgcgatacttggtgtgaattgcagaatcccgcgaaccatcgagtctttgaacgcaagttgcgcccgaagccgtcaggccgagggcacgtctgcctgggcgtcacgcatcgcgtcgctacccccacaagtcgtgtggggcagcggatgttggcctcccgtgccgtcaagcgcggccggcctaaatgagagtcctcggcgagggacgtcacgacgagtggtggttgaatgccccgactcgatttctgtcgtgccggttcccctcgctgtctccggctctacgaatgaccctcgcgcgctctctatttggagtcgcgctccgacc

SEQ ID NO: 173 - DNA, 609 bp, Pausinystalia_macroceras
tcgaatcctgcgaaacgcacgaccgtgaacttgtgtcaacagccgggcgtcgggtggcaggggagactaacccctcccttccatcccggcgccctccgcgtgcccgtcgcgcggaaaatgtaactcaaacccggcgcggaacgcgccaaggaaaaactcaataggattgtcaaaccccccgatgccccgttcgcggtgtgctagggggcgttgtgcacctgtcgtaatccaaacgactctcggcaacggatatctcggctctcgcatcgatgaagaacgtagcgaaatgcgatacttggtgtgaattgcagaatcccgcgaaccatcgagtctttgaacgcaagttgcgcccgaagccgtcaggccgagggcacgtctgcctgggcgtcacgcatcgcgtcgctacccccacaagtcgtgtggggcagcggatgttggcctcccgtgccgtcaagcgcggccggcctaaatgagagtcctcggcgagggacgtcacgacgagtggtggttgaatgccccgactcgatttctgtcgtgccggttcccctcgctgtctccggctctacgaatgaccctcgcgcgctctctatttggagtcgcgctccgacc

SEQ ID NO: 174 - DNA, 235 bp, Podophyllum_peltatum
taaaacctgcaaagcagaacgacctgagaacttgtaaaaaaacatttattgggtaagacggcggggtgcacgctcaaccgtttgcccccttggatcacgtggggtggccacgtcgctcttgtgatgtggatgcccctcgtcggatcaaacaacaaactcggcgcgatctgcgccaaggaactaaactaaaggattgagcatcttttttttatgatgttacgattccaatattcga

SEQ ID NO: 175 - DNA, 236 bp, Podophyllum_peltatum
cgaaacctgcaaaagcagaacgacccgagaacttgtaaaaaaacatttattgggtaagacggcggggtgcacgctcaaccgtttgcccccttggatcacgtggggtggccacgtcgttcttgtgatgtggatacccctcgtcggttcgaacaacaaactcggcgcgatctgcgccaaggaactaaactaaaggattgagcatcttttttttatgatgttacgattccaatattcga

SEQ ID NO: 176 - DNA, 236 bp, Podophyllum_hexandrum
caaaacctgcaaagcagaacggacccgagaacttgtaaaaaatcattttttgggtaagacggcggggtgaacgctcaaccgtctgccccttggatcatgtggggtggtcgcgtcgctcttgtgatgtggatgcccttcgtcggttcgaacaacaaactcggcgcgatctgcgccaagaaattgaactaaaggattgagcatcttcttcttgatgatgttacgattccaatatttaa

SEQ ID NO: 177 - DNA, 235 bp, Diphylleia_grayi - POD
cgaaacctgcaaagcagaacgacccgagaacttgtaaaaaaacattttttgggtaagacggtggggtgaacactcaactgtcttccctttggatcacgtgtggtggtcgcgttgctcttgtgatgtggatggccctcgtcggttcgaacaacaaactcggcgtgatctacgccaaggaactaaactaaaggattgagcatcttctttttgacgatgctacgattccaatattcga

SEQ ID NO: 178 - DNA, 235 bp, Podophyllum_sikkimense
caaaacctgcaaagcagaacgacccgagaacttgtaaaaaatcattttttgggtaagacggcggggtgaacgctcaaccgtctgccccttggatcacgtggggtggtcgcgtcgcttttgtgatgtggatacccttcgctggttcgaacaacaaactcggcacgatctgcgccaaggaattgaactaaaggattgagcatattcttcttgatgatgctacgattctaatatttaa

SEQ ID NO: 179 - DNA, 235 bp, Podophyllum_hexandrum
cgaaacctgcaaagcagaacgacccgagaacttgtaaaaaatcattttttgggtaagacggcggggtgaacgctcaatcgtctgccctttggatcacgtggggtggtcgtgtcgctcttgtgatgtggatgcccttcgtcggttcgaacaacaaactcggcgcgatctgcgccaagaaattgaattaaaggattgagcatcttcttcttgatgatgttacgatttcaatatttaa

SEQ ID NO: 180 - DNA, 371 bp, Ammandra_decasperma - S
aaggacatgatcggctgtggattcttacatccaccattttctataggaatgaagatgttcttggctcgacatagtttgttctgctctgttaggaacctaatttgtgttaggttgtaagtaaatagtacatgatggagctcgagcagaaaggattgattcgtttatcagggggaagaatctagggttagtaactatcaataagttagaccaactttgtaagtatatcctcaatatataaatcgaaaggttaaaaaaatcgaaaaatcaaagaagtttgaaaaataaaaagtaaaatttttcagaattggtaaacccatttcgatcaaaagtgtatcacaagggaatccaccgttcatattctatttctatag

SEQ ID NO: 181 - DNA, 371 bp, Attalea_allenii - S
aaggacatgatcggctgtggattcttacatccaccattttctataggaatgaagatgttcttagctcgacatagtttgttctgctctgttaggaacctaatttgtgttaggttgtaagtaaatagtacatgatggagctcgagcagaaaggattgattcgtttatcagggggaagaatctagggttagtaactattaataagttagaccaactttgtaagtatatcctcaatatataaatcgaaaggttaaaaaaatcgaaaaatcaaagaagtttgaaaaatcaaaagtaaaatttttcagaattggtaaaactatttcgatcaaaagtgtatcacaagggaatccaccgttcatattctatttctatag

SEQ ID NO: 182 - DNA, 372 bp, Phytelephas_macrocarpa - S
aaggacatgatcggctgtggattcttacatccaccattttctataggaatgaagatgttcttggctcgacatagtttgttctgctctgttaggaacctaatttgtgttaggttgtaagtaaatagtacatgatggaagctcgagcagaaaggattgattcgtttatcagggggaagaatctagggttagtaactatcaataagttagaccaactttgtaagtatatcctcaatatataaatcgaaaggttaaaaaaatcgaaaaatcaaaaaagtttgaaaaattaaaagtaaaatttttcagaattggtaaaaccatttcgatcaaaagtgtatcacaagggaatccaccgttcatatcctatttctatag

SEQ ID NO: 183 - DNA, 371 bp, Sclerosperma_mannii - S
aaggacatgatcggctgtggattcttacatccaccattttctataggaatgaagatgttcttagctcgacatagtttgttctgctctgttaggaacctaatttgtgttaggttgtaagtaaatagtacatgatggagctcgagcagaaaggattgattcgtttatcagggggaagaatctagggttagtaactatcaataagttagaccaactttgtaagtatatcctcaatatataaatcgaaaggttaaaaaaatcgaaaaatcaaagaagtttgaaaaataaaaagtaaaatttttcagaattggtaaaactatttcgatcaaaagtgtatcacaagggaatccaccgttcatattctgtttctatag

SEQ ID NO: 184 - DNA, 371 bp, Serenoa_repens
aaggacatgatcggctgtggattcttacatccaccattttctataggaatgaagatgttcttggctcgacatagtttgttctgctctgttaggaatctaatttgtgttaggttgtaagtaaatagtacatgatggagctcgagcagaaaggattgattcgtttatcagggggaagaatctagggttagtaactatcaataagttagaccaactttgtaagtatatcctcaatatataaatcgaaaggttaaaagaatcgaaaaatcaaagaagtttgaaaaataaaaagtaaaatttttcagaattggtaaaactatttcgatcaaaagtgtatcacaagggaatccaccgtgcatattctatttctatag

SEQ ID NO: 185 - DNA, 50 bp, Hypericum perforatum probe
gctttccgaaaacaaagaaagattcataaacagaaaaaaaaaaggatagg

SEQ ID NO: 186 - DNA, 49 bp, Hypericum maculatum probe
gctttccgaaaacaaagaaagattcataaacagaaaaaaaaaggatagg

SEQ ID NO: 187 - DNA, 48 bp, Hypericum pseudomaulatum probe
gctttccgaaaacaaagaaagattcataaacagaaaaaaaaggatagg

SEQ ID NO: 188 - DNA, 54 bp, Hypericum punctatum probe
gctttccgaaaacaaagcaaagaaagattcataaacagaaaaaaaaaggatagg

SEQ ID NO: 189 - DNA, 52 bp, Hypericum olympicum probe
gctttccgaaaacaaagaaagattcataaacagaaaaaaaaaaaaggatagg

SEQ ID NO: 190 - DNA, 42 bp, Hypericum perforatum probe_2
gtctggtctgataaataactgataaattgcacgagaataaag

SEQ ID NO: 191 - DNA, 42 bp, Hypericum maculatum probe_2
gtctggtctgaaaaataactgataaattgcacaagaataaag

SEQ ID NO: 192 - DNA, 42 bp, Hypericum pseudomaculatum probe_2; identical to SEQ ID NO: 190

SEQ ID NO: 193 - DNA, 42 bp, Hypericum punctatum probe_2
gtctagtttacaaaataactgattaattgcacgagaataaag

SEQ ID NO: 194 - DNA, 42 bp, Hypericum olympicum probe_2
gtctggtctgataaataactgataaattgctcgagaataaag

SEQ ID NO: 195 - DNA, 44 bp, Caulophylum robustum probe
gccttgcggcgacggcgtccccgcgggtcctaacaacaaacccg

SEQ ID NO: 196 - DNA, 44 bp, Caulophylum thalictricoides probe
gccttgcgacgacggcgtccccgtgggtcctaacaacaaacccg

SEQ ID NO: 197 - DNA, 62 bp, Echinacea atrorubens probe
gacccgtgaacatgtaaaaatactggcctttcggggaccgaaggcatttgtttcgagccttg

SEQ ID NO: 198 - DNA, 62 bp, Echinacea simulata probe
gacccgtgaacatgtataaactactggcctttcggggaccgaagcatttgtttcgagccttg

SEQ ID NO: 199 - DNA, 62 bp, Echinacea pallida probe
gacccgtgaacatgtaaaaactactggcctttcggggaccgaagcatttgtttcgagccttg

SEQ ID NO: 200 - DNA, 63 bp, Echinacea purpurea probe
gacccgtgaacatgtaaaaactactggcctttcaggggaccgaaagcatttgtttcggccttg

SEQ ID NO: 201 - DNA, 63 bp, Echinacea tennesseensis probe
gacccgtgaacatgtaaaaactacttggtctttcgggggaccgaagcatttgtttgagccttg

SEQ ID NO: 202 - DNA, 62 bp, Echinacea paradoxa probe; identical to SEQ ID NO: 199

SEQ ID NO: 203 - DNA, 40 bp, Hydrastis canadensis probe
gatctgcgaattcctactgcttatgttaaaactttccaag

SEQ ID NO: 204 - DNA, 40 bp, Caltha palustris probe
gatttgcgaattcctgttgcttatgttaaaactttccaag

SEQ ID NO: 205 - DNA, 40 bp, Psychrophila novae-zealandiae probe; identical to SEQ ID NO: 204

SEQ ID NO: 206 - DNA, 40 bp, Beesia calthifolia probe
gatctgcgaatccctgttgcttatgttaaaactttccaag

SEQ ID NO: 207 - DNA, 39 bp, Serenoa repens probe
gtttctatttataaaactagaataaatttataaaactag

SEQ ID NO: 208 - DNA, 39 bp, Trachycarpus fortunei probe
gtttctatttataaaactagaataaaaaaaggataggtg

SEQ ID NO: 209 - DNA, 39 bp, Livistona chinensis probe; identical to SEQ ID NO: 208

SEQ ID NO: 210 - DNA, 37 bp, Ammandra decasperma probe
gtttataaaactagaataaaaaaagataggataggtg

SEQ ID NO: 211 - DNA, 39 bp, Guihaia argyrata probe; identical to SEQ ID NO: 208

SEQ ID NO: 212 - DNA, 37 bp, Phytelephas aequatorialis probe; identical to SEQ ID NO: 210